(19) United States
(12) Patent Application Publication
(10) Pub. No.: US 2010/0216992 A1
(43) Pub. Date: Aug. 26, 2010

(54) MIMETIC COMPOSITIONS AND THE PRODUCTION THEREOF

(75) Inventor: Mark L. McLaughlin, Tampa, FL (US)

Correspondence Address: BRYAN CAVE LLP, 211 NORTH BROADWAY, SUITE 3600, ST. LOUIS, MO, 63102-2750, US

(73) Assignee: UNIVERSITY OF SOUTH FLORIDA, Tampa, FL (US)

(21) Appl. No.: 12/528,689

(22) PCT Filed: Feb. 27, 2008
(86) PCT No.: PCT/US08/55063

§ 371 Date: Sep. 14, 2009

Related U.S. Application Data
(60) Provisional application No. 60/891,868, filed on Feb. 27, 2007.

(51) Int. Cl. C07D 409/04; C07D 403/14

(52) U.S. Cl. 544/357

(57) ABSTRACT

The present invention generally relates to compositions including six membered heterocyclic 1,4-diaza-2-one moieties covalently bonded by a hydrazine bond and processes for producing the same. The compositions of the present invention may be utilized as peptido- and/or proteomimetics, for example,' in connection with alpha-helical proteins, like Bcl-2 proteins.

### CROSS-REFERENCE TO RELATED PARAGRAPHS

This application claims the benefit of U.S. Provisional Patent Application Ser. No. 60/891,868 filed Feb. 27, 2007, the content of which is hereby incorporated herein by reference in its entirety.

### STATEMENT OF GOVERNMENT RIGHTS

This invention was made with United States government support under grant numbers AI054246, CA118210, and GM072772 awarded by the National Institutes of Health. The United States government has certain rights in the invention.

### FIELD OF THE INVENTION

The present invention generally relates to peptido- and/or proteomimetic compositions and the production thereof. More specifically, the present invention relates to peptido- and/or proteomimetic compositions including six-membered heterocyclic 1,4-diaza-2-one moieties covalently bonded by a hydrazine bond.

### BACKGROUND OF THE INVENTION

Proteins found in the living cell are highly complex organic molecules. The structure of proteins generally involves four different architectural levels: primary, secondary, tertiary, and quaternary. The primary structure is the specific order of amino acids in the polypeptide chain. The secondary structure is the shape that the polypeptide chain forms, e.g., either an alpha-helix or a beta-pleated sheet. The tertiary structure is the specific arrangement of the secondary structure. The quaternary structure is the arrangement of all of the subunits and the nature of their contacts. See, e.g., Lubert Stryer, Biochemistry 31-3 (3rd ed., W.H. Freeman and Company 1988).

Although the core structure can be relatively similar, there are many different types of proteins carrying out a variety of different functions in biological systems. See, e.g., Randall et al., Animal Physiology Mechanisms and Adaptations 55-60, (4th ed., W.H. Freeman and Company 2001). Among other functions, proteins can provide mechanical support and coordinate motion, and can participate in enzymatic catalysis, transportation and storage, stimulation of immune protection, generation and transportation of nerve impulses, and cell growth and differentiation.

One function in which protein-protein interactions play a role is in the regulation of apoptotic pathways in cells. Programmed cell death (PCD), or apoptosis, can be triggered by a wide range of stimuli. It is a relatively highly regulated system used for the removal of unnecessary, aged, or damaged cells. Deregulation of this natural process can result in a variety of health complications ranging from cancer to autoimmune diseases. See, e.g., Chan, Clinical and Experimental Pharmacology and Physiology 31, 119-28, (2004). The apoptotic process is regulated by the interactions of pro-apoptotic (e.g., Bak, Bax, Bad, Bid) and anti-apoptotic (e.g., Bcl-2, Bcl-xL) proteins of the B-cell lymphoma-2 (Bcl-2) family. Bcl-2 is human proto-oncogene (i.e., a normal cellular gene that, when mutated or inappropriately expressed, can cause a cell to become cancerous), which is located on chromosome 18. The proteins of the Bcl-2 family are characterized by the presence of conserved sequence motifs called BH domains: BH1, BH2, BH3, BH4, which correspond to alpha-helical segments. The alpha-helix structure is the most abundant of protein secondary structures; an estimated 35% of all protein-protein interfaces involve alpha-helices.

Anti- and pro-apoptotic proteins interact with each other via BH domains. Anti-apoptotic proteins have all four, BH1-4, domains, the multinomial pro-apoptotic proteins contain BH1-3 domains, and the final group is the pro-apoptotic members that have only a BH3 domain. See, e.g., Chan, supra. The BH1-3 domains of the anti-apoptotic Bcl-2 form an elongated hydrophobic groove, which serves as the binding site for the BH3 domain of the pro-apoptotic protein. See, e.g., Chan, supra; Wendt et al., J. Med. Sci. 49, 1165-81, (2006).

The pro-apoptotic proteins are found within the cytosol and act as sensors or receivers of stress to the cell. When damage is detected, the pro-apoptotic proteins move to the surface of the mitochondria, where they have direct interaction with the mitochondrial membrane and the anti-apoptotic proteins that are located on the mitochondrial membrane surface.

Heterodimerization occurs between the pro- and anti-apoptotic proteins at the mitochondrial cell surface which can result in a neutralized or homoeostatic state for the cell. When a stress signal is received, there are more pro-apoptotic proteins present in the cell than there are anti-apoptotic proteins. This generally indicates that there are pro-apoptotic proteins remaining after all the anti-apoptotic binding sites are consumed. This allows the excess pro-apoptotic proteins to directly interact with and bind to the mitochondrial membrane. This binding disrupts the normal function of the membrane, and can lead to the formation of pores in the mitochondrial membrane. Cytochrome C, an intermediary in the apoptotic pathway, can be released from these pores, ultimately leading to cell death. See, e.g., Wendt, supra. Survival of the cell is thus determined by the balance of anti-apoptotic or the pro-apoptotic Bcl-2 members. If there is an excess of pro-apoptotic proteins present, apoptosis will be favored, and if there is an excess of anti-apoptotic proteins, resistance and survival will be favored. See, e.g., Wendt, supra.

A variety of complications are caused by the overexpression of Bcl-2. Even though the cell is triggered to release pro-apoptotic proteins, in many cases not enough of them are released to successfully interact and overtake the over expressed anti-apoptotic proteins. The “death signal” is, in essence, ignored by the cell. Overexpression can prevent apoptosis in cells that are damaged, which can lead to the continued division of the mutated cell lines and eventually cancer. See, e.g., Langenau et al., Blood 105(8), 3278-85, (2005).

Additionally, overexpression of the Bcl-2 protein can inhibit the potency of many currently available anti-cancer drugs by blocking the apoptotic pathway. See, e.g., Yin et al., JACS 127, 5463-68, (2005); Yin et al., JACS 127, 10191-6, (2005); Wendt, supra. Thus, agents that directly mimic the death-promoting region BH3 domain of the pro-apoptotic subfamily of Bcl-2 are of potential therapeutic value. See, e.g., Wendt, supra.

While there has been moderate success in the formation of small peptidomimetics that reproduce certain features of relatively short peptides, less progress has been made in the search for structures that mimic larger regions of the protein surface, such as the alpha-helix.

As noted above, the alpha-helix is an abundant secondary structure in proteins that participates in a variety of biological functions such as in the regulation of a variety of pathways, including apoptotic pathways in cancer cells discussed above. In alpha-helix protein complexes, interactions are frequently found along one face of the helix, involving side chains from the ith, ith+3, ith+4, and ith+7 positions.

Several approaches have been explored in an attempt to provide improved peptido- and proteomimetics. Some approaches have involved the production of mimetics which impart or stabilize alpha-helicity to proteins or peptides (see, e.g., Kahn et al., U.S. Pat. Nos. 5,840,833 and 5,859,184, hereby incorporated by reference herein), while other approaches have involved the production of mimetics which interact with certain regions and/or positions of the alpha-helix. For example, in U.S. Pat. No. 6,858,600 (hereby incorporated by reference herein), Hamilton et al. disclose libraries of proteomimetic compounds based on a tris-functionalized 3,2′,2″-terphenyl derivative that interact with the ith, ith+3, and ith+7 positions of the alpha-helix.

Although several peptido- and proteomimetics of varying form and function are known, there are limitations to their effectiveness including, for example, relatively low hydrophilicity, poor cell membrane permeability, and/or relatively small size. Medications and therapeutic agents including peptido- and/or proteomimetics, for example, typically benefit from having readily soluble active ingredients. Accordingly, a need remains for improved peptido- and/or proteomimetic compositions suitable for understanding, mediating, and/or regulating the myriad of interactions involved in biological functions.

### SUMMARY OF THE INVENTION

Among the various aspects of the present invention is the provision of compositions including six-membered heterocyclic 1,4-diaza-2-one moieties covalently bonded by a hydrazine bond and processes for preparing the same. The compositions of the present invention exhibit peptidomimetic and proteomimetic characteristics and, in particular, mimic alpha-helical proteins and regions thereof, such as the proteins of the B-cell lymphoma-2 (Bcl-2) family.

Briefly, therefore, the present invention is directed to a composition having the formula Z₁—(W₁)ₙ—P₁-P₂—(W₂)ₘ—Z₂ wherein

P₁ and P₂ are six-membered heterocyclic 1,4-diaza-2-one moieties covalently bonded by a hydrazine bond;

each W₁ and W₂ is independently a six-membered carbocyclic or heterocyclic ring;

Z₁ and Z₂ are independently hydrogen, hydrocarbyl, substituted hydrocarbyl, heterocyclo, acyl, amino, protected amino, sulfonyl, phosphoryl, or a linker moiety attached to a solid support; and

n is 0 to 10 and m is 0 to 10 provided, however, the sum of n and m is 1 to 10.

The present invention is also directed to a process for the preparation of a composition containing two, six-membered heterocyclic 1,4-diaza-2-one moieties covalently bonded by a hydrazine bond, the process comprising treating a six-membered heterocyclic 1,4-diaza-4′-amino-2-one with a monoester (44a) or a diacid (44b) in the presence of an activating agent, wherein

the monoester (44a) and the diacid (44b) correspond to Formulae (44a) or (44b):

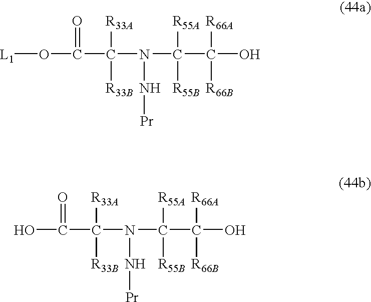

L₁ is hydrocarbyl or substituted hydrocarbyl;

Pr is an amino protecting group;

R_{3A}, R_{3B}, R_{33A}, and R_{33B} are independently hydrogen, substituted or unsubstituted hydrocarbyl, heterocyclo, alkoxy, ester, thioester, thioether, amino, or amido;

R_{55A} and R_{55B} are independently hydrogen, hydrocarbyl, substituted hydrocarbyl, or together with the carbon atom to which they are attached form a five- or six-membered ring;

R_{66A} is hydrogen, sulfonyl, or phosphoryl, or together with R_{66B} forms ═O; and

R_{66B} is hydrogen or together with R_{66A} forms ═O.

Other objects and features will be in part apparent and in part pointed out hereinafter.

### BRIEF DESCRIPTION OF THE DRAWINGS

FIG. 1 is a graph depicting the ability of a proteomimetic composition produced as described herein to interrupt GST-Bcl-x_{L} binding to Bak BH3-F (FPA) (see Example 8).

### DETAILED DESCRIPTION OF THE INVENTION

Among other things, the present invention enables the preparation of peptido- and/or proteomimetic compositions. The compositions of the present invention represent chemical cages or scaffolds in which two or three (or more) six-membered heterocyclic 1,4-diaza-2-one moieties are covalently bonded by a hydrazine bond (i.e., N—N). Each individual six-membered heterocyclic 1,4-diaza-2-one moiety, and other six-membered carbocyclic and heterocyclic moieties described herein, may be independently substituted at the 3-, 5-, and 6-positions of the heterocyclic ring. Preferably, the six-membered heterocyclic 1,4-diaza-2-one moieties forming the chemical cages or scaffolds are substituted with substituents which mimic natural substituents on alpha carbons (from the amino acids) of alpha-helical proteins (e.g., at the 3-position).

The compositions of the present invention are also preferably sterically and electronically similar to the steric and/or electronic physiochemical characteristics of substituents on alpha carbons in natural alpha-helical proteins. While not necessarily identical to the natural substituents, substituents which are sterically and electronically similar to the natural substituents generally promote the binding of synthetic compositions of the present invention to alpha-helical protein binding sites. In a particular embodiment, the mimetic compositions of the present invention carry one or more amino acid side chain substituents (or derivatives thereof) in positions which mimic the ith, ith+3, ith+4, and ith+7 positions of an alpha-helix.

Advantageously, the compositions of the present invention are relatively highly soluble as compared to known peptido- and proteomimetics, such as those described by Hamilton et al. in U.S. Pat. No. 6,858,600. By way of example, the compositions of the present invention include a relatively hydrophilic core, yet conserve the hydrophobic side chains. Among other properties, the high solubility of active ingredients can play an important role in the development of medications and therapeutic agents, as water comprises a large percentage of mammalian body weight.

Mimetic Compositions

The present invention offers particular advantages in the preparation of compositions including at least two, six-membered heterocyclic 1,4-diaza-2-one moieties covalently bonded by a hydrazine bond. If desired, the compositions can include as many as twelve, or more, six-membered heterocyclic 1,4-diaza-2-one moieties covalently bonded by a hydrazine bond, or other six-membered carbocyclic or heterocyclic rings.

In one embodiment, the compositions of the present invention have the formula Z₁—(W₁)ₙ—P₁-P₂—(W₂)ₘ—Z₂ wherein

P₁ and P₂ are six-membered heterocyclic 1,4-diaza-2-one moieties covalently bonded by a hydrazine bond;

each W₁ and W₂ is independently a six-membered carbocyclic or heterocyclic ring;

Z₁ and Z₂ are independently hydrogen, hydrocarbyl, substituted hydrocarbyl, heterocyclo, acyl, amino, protected amino, sulfonyl, phosphoryl, or a linker moiety attached to a solid support; and

n is 0 to 10 and m is 0 to 10 provided, however, the sum of n and m is 1 to 10.

The P₁ and P₂ Moieties

As noted above, P₁ and P₂ are six-membered heterocyclic 1,4-diaza-2-one moieties covalently bonded by a hydrazine bond. In one embodiment, the six-membered heterocyclic 1,4-diaza-2-one moieties covalently bonded by a hydrazine bond (i.e., P₁ and P₂) correspond to Formula (50):

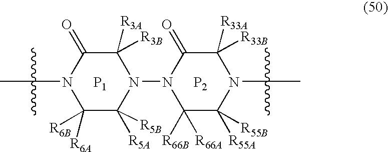

wherein

R_{3A}, R_{3B}, R_{33A}, and R_{33B} are independently hydrogen, substituted or unsubstituted hydrocarbyl (e.g., substituted or unsubstituted aryl, substituted or unsubstituted alkylenearyl, substituted or unsubstituted alkylaryl, alkanol, alkanoic acid, substituted or unsubstituted alkylene amine, substituted or unsubstituted alkylene amide, substituted or unsubstituted alkyleneguanidine), heterocyclo, alkoxy, ester, thioester, thioether, amino (e.g., substituted or unsubstituted amines such as alkylamine and dialkylamine), or amido (e.g., substituted or unsubstituted amide);

R_{5A}, R_{5B}, R_{55A}, and R_{55B} are independently hydrogen, hydrocarbyl, substituted hydrocarbyl, or together with the carbon atom to which they are attached form a five- or six-membered ring;

R_{6A} is hydrogen, sulfonyl, or phosphoryl, or together with R_{6B} forms ═O;

R_{6B} is hydrogen or together with R_{6A} forms ═O;

R_{66A} is hydrogen, sulfonyl, or phosphoryl, or together with R_{66B} forms ═O; and

R_{66B} is hydrogen or together with R_{66A} forms ═O;

As depicted in Formula (50), the nitrogen atoms of the six-membered heterocyclic 1,4-diaza-2-one moieties, i.e., P₁ and P₂, occupy the 1-position and the 4-position of each of the P₁ and P₂ rings, the carbon atom occupying the 2-position of each of the P₁ and P₂ rings bears a keto (i.e., ═O) substituent, and the nitrogen atom in the 4-position of P₁ is covalently bonded to the nitrogen atom in the 1-position of P₂. As described, the peptido- and/or proteomimetic compositions of the present invention include at least one (n+m=1), and possibly as many as 10 (n+m=10) additional six-membered rings discussed below (i.e., W₁ and/or W₂); these additional six-membered rings, however, are not necessarily 1,4-diaza-2-ones. For example, they may be carbocyclic or other heterocycle. For ease of discussion herein, the numbering system of each additional six-membered ring present shall be assumed to be similar regardless of the substitution pattern. That is, for purposes of discussion, it is understood that when two such rings are bonded, the 1-ring position of one is bonded to the 4-ring position of the other. This is illustrated below:

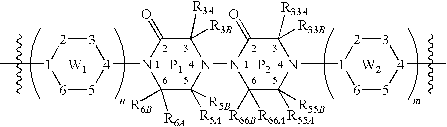

Accordingly, the various substituents connected to the atom occupying the 3-position are the R₃ substituents (e.g., R_{3A}, R_{3B}, R_{33A}, R_{33B}, R_{333A}, and R_{333B}, and so on); the various substituents connected to the atom occupying the 5-position are the R₅ substituents (e.g., R_{5A}, R_{5B}, R_{55A}, R_{55B}, R_{555A}, and R_{555B}, and so on); and the various substituents connected to the atom occupying the 6-position are the R₆ substituents (e.g., R_{6A}, R_{6B}, R_{66A}, R_{66B}, R_{666A}, R_{666B}, and so on

As noted above, depending on the value for m and n there will be at least one and possibly as many as 10 additional six-membered rings attached to the 1-position and/or the 4-position of the —P₁-P₂— moiety in the manner shown above. Accordingly, these rings may be substituted at their respective 3-positions with further R₃ substituents; that is, R_{333A} and R_{333B}; R_{3333A} and R_{3333B}; R_{33333A} and R_{33333B}; R_{333333A} and R_{333333B}; R_{3333333A} and R_{3333333B}; R_{33333333A} and R_{33333333B}; R_{333333333A} and R_{333333333B}; R_{3333333333A} and R_{3333333333B}; R_{33333333333A} and R_{33333333333B}; and R_{333333333333A} and R_{333333333333B}. It will be understood that the additional six-membered rings will be similarly substituted with R groups at the 5-position and the 6-position of each six-membered ring, but a recitiation of each possible R₅ and R₆ substituent is not included in the interest of brevity.

In general, the R₃ substituents (e.g., R_{3A}, R_{3B}, R_{33A}, R_{33B}, R_{333A}, R_{333B}, and so on) on the six-membered heterocyclic and carbocyclic moieties described herein are selected to reproduce the recognition properties of the amino acid side chains of proteins, such as alpha-helical proteins (e.g., proteins of the B-cell lymphoma-2 (Bcl-2) family such as, for instance, the death-promoting region BH3 domain of the pro-apoptotic subfamily of Bcl-2, or blocking the interaction of p-53 with MDM2 or MDMX). The various definitions for R_{3A}, R_{3B}, R_{33A}, and R_{33B}, therefore, generally correspond to amino acid side chain moieties present in any naturally occurring or non-naturally occurring amino acid. This includes naturally occurring alpha-amino acids, as well as non-naturally occurring amino acids such as beta-amino acids and gamma-amino acids that are commonly used by those in the field of peptide chemistry when preparing synthetic analogues of naturally occurring peptides, including D and L forms. By way of example, the definitions for the R₃ substitutents (e.g., R_{3A}, R_{3B}, R_{33A}, and R_{33B}, etc.) are intended to encompass the amino acid side chain moieties from the proteogenic L-amino acids and esters thereof (i.e., from the 20 amino acids commonly incorporated into proteins), as well as D-amino acids and esters thereof and non-proteogenic amino acids and esters thereof (i.e., metabolites or analogues of proteogenic amino acids).

By way of example, the various definitions for the R₃ substituents (e.g., R_{3A}, R_{3B}, R_{33A}, R_{33B}, R_{333A}, R_{333B}, and so on) at the C(3) position of the heterocyclic ring may encompass one or more of the amino acid side chain moieties identified in Table 1.

TABLE 1

| C(3) Substituent(s) | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₂ | Lysine |
| —(CH₂)₃NHC(═NH)NH₂ | Arginine |
| 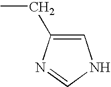 | Histidine |
| —CH₂COOH | Aspartic Acid |
| —CH₂CH₂COOH | Glutamic Acid |
| —CH₂CONH₂ | Asparagine |
| —CH₂CH₂CONH₂ | Glutamine |
| 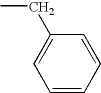 | Phenylalanine |
| 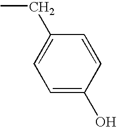 | Tyrosine |
| 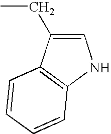 | Tryptophan |
| —CH₂SH | Cysteine |
| —CH₂CH₂SCH₃ | Methionine |
| —CH₂OH | Serine |
| —CH(OH)CH₃ | Threonine |

For convenience purposes, only the unionized form of certain of the amino acid side chain moieties has been shown in Table 1. It is contemplated, however, that the amino acid side chain moieties illustrated in Table 1 may be utilized in the anionic, or conjugate base, form, in combination with a cation, or protonated with a counterion. Suitable cations and counterions are described in further detail below.

Other naturally occurring amino acid side chain moieties encompassed by the various definitions of the R₃ substituents (e.g., R_{3A}, R_{3B}, R_{33A}, R_{33B}, R_{333A}, R_{333B}, and so on) include, but are not limited to, the amino acid side chain moieties of 3,5-dibromotyrosine, 3,5-diiodotyrosine, hydroxylysine, γ-carboxyglutamate, phosphotyrosine, and phosphoserine. In addition, glycosylated amino acid side chain moieties may also be used in the practice of this invention, including (but not limited to) glycosylated serine, threonine, and asparagine.

In addition to naturally occurring amino acid side chain moieties, the various definitions of the R₃ substituents (e.g., R_{3A}, R_{3B}, R_{33A}, R_{33B}, R_{333A}, R_{333B}, and so on) are intended to encompass amino acid side chain moieties from various amino acid derivatives.

Generally speaking, the amino acid derivatives include one or more modifications and/or variations to naturally occurring amino acid side chain moieties. For example, the amino acid side chain moieties of alanine, valine, leucine, isoleucine and phenylalanine may generally be classified as alkyl (e.g., C₁ to C₁₂), aryl (e.g., C₆ to C₁₂) or aralkyl (e.g., C₇ to C₁₂) moieties. Derivatives of amino acid side chain moieties include other straight chain or branched, cyclic or noncyclic, substituted or unsubstituted, saturated or unsaturated lower chain alkyl, aryl, or aralkyl moieties. Thus, in various embodiments, the R₃ substituents (e.g., R_{3A}, R_{3B}, R_{33A}, R_{33B}, R_{333A}, R_{333B}, and so on) are independently C₁ to C₁₂ alkyl, a C₆ to C₁₂ aryl, and a C₇ to C₁₂ aralkyl; more preferably in this embodiment, a C₁ to C₇ alkyl, a C₆ to C₁₀ aryl, and a C₇ to C₁₁ aralkyl.

The various definitions of the R₃ substituents (e.g., R_{3A}, R_{3B}, R_{33A}, R_{33B}, R_{333A}, R_{333B}, and so on) are also intended to encompass, for example, substituted derivatives of alkyl (e.g., C₁ to C₁₂), aryl (e.g., C₆ to C₁₂) or aralkyl (e.g., C₇ to C₁₂) moieties. Exemplary substituents which may be found on such moieties include, but are not limited to, one or more of the following chemical moieties: —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), wherein each occurrence of R may be hydrocarbyl or substituted hydrocarbyl (e.g., substituted or unsubstituted alkyl, substituted or unsubstituted aryl, or substituted or unsubstituted aralkyl).

Moreover, the various definitions of the R₃ substituents (e.g., R_{3A}, R_{3B}, R_{33A}, R_{33B}, R_{333A}, R_{333B}, and so on) may also encompass one or more bicyclic (e.g., napthyl) or heterocyclic moieties such as thiophene, pyrrole, furan, imidazole, oxazole, thiazole, pyrazole, 3-pyrroline, pyrrolidine, pyridine, pyrimidine, purine, quinoline, isoquinoline, and carbazole. Additionally or alternatively, the various definitions of the R₃ substituents (e.g., R_{3A}, R_{3B}, R_{33A}, R_{33B}, R_{333A}, R_{333B}, and so on) may include heteroalkyl derivatives of the alkyl and aralkyl moieties, including (but not limited to) alkyl and aralkyl phosphonates and silanes.

As noted above, while the R₃ substituents may be selected from hydrogen, substituted or unsubstituted hydrocarbyl, heterocyclo, alkoxy, ester, thioester, thioether, amino, or amido, in one particular embodiment the R₃ substituents (e.g., R_{3A}, R_{3B}, R_{33A}, R_{33B}, R_{333A}, R_{333B}, and so on) are independently hydrogen, hydrocarbyl, or substituted hydrocarbyl. In various embodiments, at least one of the R₃ substituents (e.g., R_{3A}, R_{3B}, R_{33A}, R_{33B}, R_{333A}, R_{333B}, and so on) is other than hydrogen.

Where one or more of the R₃ substituents (e.g., R_{3A}, R_{3B}, R_{33A}, R_{33B}, R_{333A}, R_{333B}, and so on) are hydrocarbyl, for example, they may be independently alkyl, alkenyl, alkynyl, aryl, alkaryl, or aralkyl. Typically, such substituents contain from 1 to 20 carbon atoms and may be linear, branched, or cyclic. By way of example, the R₃ substituents may be selected from methyl, ethyl, n-propyl, cyclopropyl, isopropyl, n-butyl, cyclobutyl, isobutyl, s-butyl, n-pentyl, isopentyl, cyclopentyl, n-hexyl, isohexyl, cyclohexyl, benzyl, phenyl, and napthyl. Such moieties may correspond to particular amino acid side chain moieties including, for example, alanine, valine, leucine, isoleucine, and phenylalanine, among others.

Where one or more of the R₃ substituents (e.g., R_{3A}, R_{3B}, R_{33A}, R_{33B}, R_{333A}, R_{333B}, and so on) are substituted hydrocarbyl, for example, they may be independently substituted alkyl, substituted alkenyl, substituted alkynyl, substituted aryl, substituted alkaryl, or substituted aralkyl. Similar to the hydrocarbyl moieties described in the preceding paragraph, these substituents may contain 1 to 20 carbon atoms and may be linear, branched, or cyclic; one or more hydrogen atoms of the substituted hydrocarbyl moieties, however, are replaced with a different substituent such as, for example, —OH, —OR, —COOH, —COOR, —CONH₂, —NH₂, —NHR, —NRR, —SH, —SR, —SO₂R, —SO₂H, —SOR, heterocyclo, and halo (including F, Cl, Br and I), among others, wherein each occurrence of R may be hydrocarbyl or substituted hydrocarbyl (e.g., substituted or unsubstituted alkyl, substituted or unsubstituted aryl, or substituted or unsubstituted aralkyl). Such substituted hydrocarbyl moieties may correspond to particular amino acid side chain moieties including, for example, lysine, arginine, histidine, aspartic acid, glutamic acid, asparagine, glutamine, phenylalanine, tyrosine, tryptophan, cysteine, methionine, serine, and threonine, among others.

Thus, the R₃ substituents (e.g., R_{3A}, R_{3B}, R_{33A}, R_{33B}, R_{333A}, R_{333B}, and so on) may be selected from hydrogen, substituted or unsubstituted hydrocarbyl (including, e.g., substituted or unsubstituted alkyl, alkenyl, or alkynyl, substituted or unsubstituted aryl (e.g., benzyl, hydroxybenzyl, napthyl, and the like), substituted or unsubstituted alkylenearyl (e.g., alkylenephenyl), substituted or unsubstituted alkylaryl (e.g., alkylphenyl), alkanol (e.g., a C₁ to C₆ alkanol such as hydroxymethyl, hydroxyethyl, hydroxypropyl, hydroxybutyl, hydroxypentyl, or hydroxyhexyl), alkanoic acid (e.g., a C₁ to C₆ alkanoic acid including, for example, a carboxylic acid without further substitution), substituted or unsubstituted alkylene amine (e.g., a C₁ to C₆ alkylene amine, including, for example, saturated ring systems containing nitrogen groups), substituted or unsubstituted alkylene amide (e.g., a C₁ to C₆ alkylene substituted on the amine groups of the amide, for example, with alkyl groups) alkyleneguanidine (e.g., a C₁ to C₆ alkyleneguanidine)), heterocyclo, alkoxy, ester (including, for example, an alkyl, alkenyl, or aryl ester, wherein said ester group comprises a C₁ to C₆ alkyl or aryl (e.g., phenyl or benzyl)), thioester (e.g., a C₁ to C₆ alkyl or C₁ to C₆ alkylene thioester), thioether (e.g., a C₁ to C₆ alkyl or C₁ to C₆ alkylene thioether such as methyl ethylene (methionine) thioether), amines (including, for example, a substituted or unsubstituted amine (e.g., —NH₂, an alkylamine or dialkylamine such as a C₁ to C₆ alkylamine or a C₁ to C₆ dialkylamine)), and amides (including, for example, substituted or unsubstituted amides).

In one embodiment, the R₃ substituents (e.g., R_{3A}, R_{3B}, R_{33A}, R_{33B}, R_{333A}, R_{333B}, and so on) are independently hydrogen, alkyl, benzyl, phenyl, napthyl, hydroxybenzyl, alkanol, alkyl alkylene thioether, alkanoic acid, alkylamide, alkyleneamine, or alkyleneguanidine; more preferably in this embodiment, hydrogen, C₁ to C₆ alkyl, benzyl, phenyl, napthyl, hydroxybenzyl, C₁ to C₆ alkanol, alkyl alkylene thioether, C₁ to C₆ alkanoic acid, C₁ to C₆ alkylamide, C₁ to C₆ alkyleneamine, or C₁ to C₆ alkyleneguanidine. In another embodiment, the R₃ substituents (e.g., R_{3A}, R_{3B}, R_{33A}, R_{33B}, R_{333A}, R_{333B}, and so on) are independently hydrogen, alkanoic acid, alkanol, alkyl, substituted alkyl, alkylene amide, alkylene amine, aralkyl, substituted aralkyl, or heteroaralkyl; more preferably in this embodiment, hydrogen, C₁ to C₆ alkanoic acid, C₁ to C₆ alkanol, C₁ to C₆ alkyl, substituted C₁ to C₆ alkyl, C₁ to C₆ alkylene amide, C₁ to C₆ alkylene amine, aralkyl, substituted aralkyl, or heteroaralkyl.

Additionally, it will be understood that the two R₃ substituents, R_{3A} and R_{3B}, etc., at the 3-position of each six-membered heterocyclic 1,4-diaza-2-one moiety may be the same or different. For instance, one of R_{3A} and R_{3B} may be hydrogen and the other of R_{3A} and R_{3B} may be hydrocarbyl or substituted hydrocarbyl. Alternatively, both R_{3A} and R_{3B} may be the same and may be hydrocarbyl (e.g., R_{3A} and R_{3B} are the same and are selected from linear or branched alkyl moieties) or substituted hydrocarbyl. By way of further example, one of R_{3A} and R_{3B} may be hydrogen and the other of R_{3A} and R_{3B} may be selected from the amino acid side chain moieties shown in Table 1. Or, R_{3A} and R_{3B} may be the same and selected from the amino acid side chain moieties shown in Table 1. It will also be understood that these examples further apply to the R_{33A} and R_{33B} substituents, the R_{333A} and R_{333B} substituents, and so on, as described above. In one embodiment, for example, at least one of R_{3A}, R_{3B}, R_{33A}, and R_{33B} is other than hydrogen. In another embodiment, for example, at least one of R_{3A}, R_{3B}, R_{33A}, R_{33B}, R_{333A}, and R_{333B} is other than hydrogen.

In addition to R_{3A}, R_{3B}, R_{33A}, and R_{33B} described above, the six-membered heterocyclic 1,4-diaza-2-one moieties covalently bonded by a hydrazine bond corresponding to Formula (50) also carry the substituents R_{5A}, R_{5B}, R_{55A}, and R_{55B}, which are independently hydrogen, hydrocarbyl, substituted hydrocarbyl, or together with the carbon atom to which they are attached form a five- or six-membered ring. Thus, for example, R_{5A}, R_{5B}, R_{55A}, and R_{55B} may be hydrogen, alkyl, alkenyl, or alkynyl. Alternatively, R_{5A}, R_{5B}, R_{55A}, and R_{55B}, together with the carbon atom to which they are attached, may form a five- or six-membered spiro system, which may be substituted or unsubstituted. Typically, R_{5A}, R_{5B}, R_{55A}, and R_{55B} are independently hydrogen or alkyl (e.g., methyl, ethyl, propyl, butyl, etc.). Preferably, the R_{5A} and R_{5B} substituents and the R_{55A} and R_{55B} substituents are the same (e.g., each R_{5A} and R_{5B} is alkyl or hydrogen and/or each R_{55A} and R_{55B} is alkyl or hydrogen). In one embodiment, each R_{5A}, R_{5B}, R_{55A}, and R_{55B} is hydrogen.

The six-membered heterocyclic 1,4-diaza-2-one moieties covalently bonded by a hydrazine bond corresponding to Formula (50) also carry the substituents R_{6A}, R_{6B}, R_{66A}, and R_{66B}. R_{6A} is hydrogen, sulfonyl, or phosphoryl, or together with R_{6B} forms ═O, and R_{6B} is hydrogen or together with R_{6A} forms ═O. Similarly, R_{66A} is hydrogen, sulfonyl, or phosphoryl, or together with R_{66B} forms ═O, and R_{66B} is hydrogen or together with R_{66A} forms ═O. In one embodiment, R_{6A} and R_{6B} are hydrogen or together form ═O, and R_{66A} and R_{66B} are hydrogen or together form ═O; more preferably in this embodiment R_{6A} and R_{6B} together form ═O, R_{66A} and R_{66B} together form ═O.

In combination, among certain of the preferred embodiments are six-membered heterocyclic 1,4-diaza-2-one moieties covalently bonded by a hydrazine bond corresponding to Formula (50) wherein:

R_{3A}, R_{3B}, R_{33A}, and R_{33B} are independently hydrogen, hydrocarbyl, or substituted hydrocarbyl (such as, for example, unsubstituted C₁-C₈ alkyl (e.g., methyl, ethyl, propyl, butyl, etc.), substituted or unsubstituted aryl (e.g., benzyl, phenyl, napthyl, hydroxybenzyl, etc.) or substituted C₁-C₆ alkyl (e.g., alkanol, alkyl alkylene thioether, alkanoic acid, alkyleneamide, alkyleneamine, alkyleneguanidine, etc.));

R_{5A}, R_{5B}, R_{55A}, and R_{55B} are hydrogen;

R_{6A} and R_{6B} together form ═O; and

R_{66A} and R_{66B} together form ═O.

According to other preferred embodiments are six-membered heterocyclic 1,4-diaza-2-one moieties covalently bonded by a hydrazine bond corresponding to Formula (50) wherein:

R_{3A}, R_{3B}, R_{33A}, and R_{33B} are independently hydrogen, alkanoic acid, alkanol, alkyl, substituted alkyl, alkylene amide, alkylene amine, aralkyl, substituted aralkyl, or heteroaralkyl;

R_{5A}, R_{5B}, R_{55A}, and R_{55B} are hydrogen;

R_{6A} and R_{6B} together form ═O; and

R_{66A} and R_{66B} together form ═O.

According to the latter embodiment (i.e., when R_{6A} and R_{6B} together form ═O and R_{66A} and R_{66B} together form ═O), therefore, the —P₁-P₂— moiety corresponds to two six-membered heterocyclic 1,4-diaza-2,6-one moieties covalently bonded by a hydrazine bond.

In accordance with these embodiments, therefore, the six-membered heterocyclic 1,4-diaza-2-one moieties covalently bonded by a hydrazine bond (i.e., P₁ and P₂) corresponds to Formula (500):

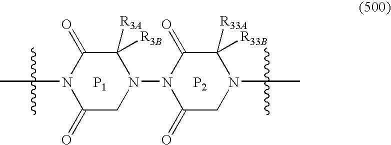

wherein R_{3A}, R_{3B}, R_{33A}, and R_{33B} are as defined above in connection with Formula (50). Thus, for example, when the six-membered heterocyclic 1,4-diaza-2-one moieties covalently bonded by a hydrazine bond (i.e., P₁ and P₂) corresponds to Formula (500), R_{3A}, R_{3B}, R_{33A}, and R_{33B} may be independently hydrogen, hydrocarbyl, or substituted hydrocarbyl. In a particular embodiment in which the six-membered heterocyclic 1,4-diaza-2-one moieties covalently bonded by a hydrazine bond (i.e., P₁ and P₂) corresponds to Formula (500), R_{3A}, R_{3B}, R_{33A}, and R_{33B} are independently hydrogen, alkanoic acid, alkanol, alkyl, substituted alkyl, alkylene amide, alkylene amine, aralkyl, substituted aralkyl, or heteroaralkyl. According to various embodiments in which when the six-membered heterocyclic 1,4-diaza-2-one moieties covalently bonded by a hydrazine bond (i.e., P₁ and P₂) corresponds to Formula (500), at least one of R_{3A}, R_{3B}, R_{33A}, and R_{33B} is other than hydrogen.

The W₁ and W₂ Moieties

As further noted in connection with formula Z₁—(W₁)_{n—P}₁-P₂—(W₂)_{m—Z}₂, each W₁ and W₂ is independently a six-membered carbocyclic ring or heterocyclic ring. In general, the number of W₁ and/or W₂ moieties present in the formula varies based on the value of n and m. In general, n (thus, the number of W₁ moieties) is 0 to 10 and m (thus, the number of W₂ moieties) is 0 to 10, provided, however, the sum of n and m is 1 to 10. Accordingly, at least one W₁ moiety or one W₂ moiety is present in the formula. By way of example, the sum of n and m may be 1, 2, 3, 4, 5, 6, 7, 8, 9, or 10; thus, the formula may include one, two, three, four, five, six, seven, eight, nine, or ten W₁ or W₂ moieties or a combination or W₁ and W₂ moieties. The sum of n and m is preferably less than 8 (e.g., 1, 2, 3, 4, 5, 6, or 7), and typically less than 5 (e.g., 1, 2, 3, or 4). In one embodiment, the sum of n and m is 1 or 2. In another embodiment, the sum of n and m is 1, 2, 3, 4, or 5. In another embodiment, the sum of n and m is 1, 2, or 3.

A variety of six-membered carbocyclic or heterocyclic rings may occupy the W₁ and/or W₂ position. For instance, the W₁ and/or W₂ moiety may be a six-membered carbocyclic or heterocyclic ring that enhances certain interactions (e.g., electrostatic, hydrophobic, and/or mimetic interactions) between the composition and a target protein. Additionally or alternatively, the W₁ and/or W₂ moiety may be a six-membered carbocyclic or heterocyclic ring that affects the solubility or other pharmacokinetic property of the composition, and/or that affects the ability of the composition to penetrate cellular membranes.

By way of example, W₁ and/or W₂ could generally correspond to a six-membered heterocyclic 1,4-diaza-2-one moiety as described above in connection with P₁ and P₂. In one embodiment, each W₁ and W₂ corresponds to Formula (60):

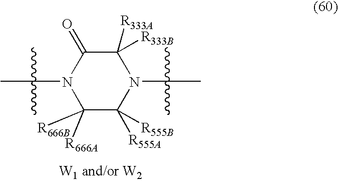

wherein R_{333A} and R_{333B} are defined in connection with the R_{3A}, R_{3B}, R_{33A}, and R_{33B} substituents of Formula (50); R_{555A} and R_{555B} are defined in connection with the R_{5A}, R_{5B}, R_{55A}, and R_{55B} substituents of Formula (50); and R_{666A} and R_{666B} are defined in connection with the R_{6A}, R_{6B}, R_{66A}, and R_{66B} substituents of Formula (50).

Thus, for example, R_{333A} and R_{333B} may be independently hydrogen, hydrocarbyl, or substituted hydrocarbyl (such as, for example, unsubstituted C₁-C₈ alkyl (e.g., methyl, ethyl, propyl, butyl, etc.), substituted or unsubstituted aryl (e.g., benzyl, phenyl, napthyl, hydroxybenzyl, etc.) or substituted C₁-C₆ alkyl (e.g., alkanol, alkyl alkylene thioether, alkanoic acid, alkyleneamide, alkyleneamine, alkyleneguanidine, etc.));

R_{555A} and R_{555B} may be independently hydrogen, hydrocarbyl, substituted hydrocarbyl, or together with the carbon atom to which they are attached form a five- or six-membered ring;

R_{666A} may be hydrogen, sulfonyl, or phosphoryl, or together with R_{666B} form ═O; and

R_{666B} may be hydrogen or together with R_{666A} form ═O.

In combination, among certain of the preferred embodiments are six-membered heterocyclic rings (i.e., W₁ and/or W₂) corresponding to Formula (60) wherein:

R_{333A} and R_{333B} are independently hydrogen, alkyl, benzyl, phenyl, napthyl, hydroxybenzyl, alkanol, alkyl alkylene thioether, alkanoic acid, alkylamide, alkyleneamine, and alkyleneguanidine;

R_{555A} and R_{555B} are hydrogen; and

R_{666A} and R_{666B} together form ═O.

According to other preferred embodiments are six-membered heterocyclic rings (i.e., W₁ and/or W₂) corresponding to Formula (60) wherein:

R_{333A} and R_{333B} are independently hydrogen, alkanoic acid, alkanol, alkyl, substituted alkyl, alkylene amide, alkylene amine, aralkyl, substituted aralkyl, or heteroaralkyl;

R_{555A} and R_{555B} are hydrogen; and

R_{666A} and R_{666B} together form ═O.

In accordance with these embodiments, therefore, the six-membered heterocyclic rings (i.e., W₁ and/or W₂) correspond to Formula (600):

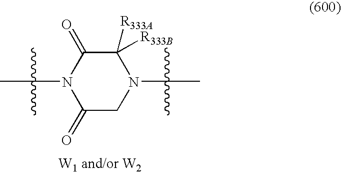

wherein R_{333A} and R_{333B} are defined in connection with the R_{3A}, R_{3B}, R_{33A}, and R_{33B} substituents of Formula (50). Thus, for example, when the six-membered heterocyclic rings (i.e., W₁ and/or W₂) correspond to Formula (600), R_{333A} and R_{333B} may be independently hydrogen, hydrocarbyl, or substituted hydrocarbyl. In a particular embodiment in which the six-membered rings (i.e., W₁ and/or W₂) correspond to Formula (600), R_{333A} and R_{333B} are independently hydrogen, alkanoic acid, alkanol, alkyl, substituted alkyl, alkylene amide, alkylene amine, aralkyl, substituted aralkyl, or heteroaralkyl. According to various embodiments in which the six-membered rings (i.e., W₁ and/or W₂) correspond to Formula (600), at least one of R_{333A} and R_{333B} is other than hydrogen.

Alternatively, W₁ and/or W₂ could generally correspond to one or more of the functionalized terphenyls and related six-membered carbocyclic and heterocyclic compounds which comprise the proteomimetic subunits described in U.S. Pat. No. 6,858,600 to Hamilton et al. (hereby incorporated by reference herein).

Regardless of the structure of and/or substituents carried by the six-membered carbocyclic or heterocyclic rings, the W₁ and/or W₂ moieties may be attached to the P₁ and/or P₂ moieties by way of a covalent bond or by way of a non-covalent interaction such as hydrogen bonding, ionic bonding, Van der Waals forces, hydrophobic interactions, and the like. Preferably, the W₁ and/or W₂ moieties are covalently bonded to the P₁ and/or P₂ moieties. Thus, for the example the W₁ and/or W₂ moiety could be covalently bonded to P₁ and/or P₂ by a hydrazine bond or some other connecting bond, such as an amide group (e.g., —NH—C(═O)—) or an ester group (e.g., —O—C(═O)—). Typically, however, the W₁ and/or W₂ moieties are covalently bonded to the P₁ and/or P₂ moieties by a hydrazine bond.

Generally speaking, compositions including more than six or seven W₁ and/or W₂ moieties corresponding to Formula (60) or (600) (i.e., wherein the sum of n and m is 6, 7, 8, 9, or 10) and carrying the various substituents noted above (e.g., R_{333A}, R_{333B}, R_{555A}, R_{555B}, R_{666A}, and R_{666B}) may be more difficult to synthesize and/or there may be less likelihood in finding a suitable peptide registry match. In the embodiments in which W₁ and/or W₂ correspond to a six-membered heterocyclic 1,4-diaza-2-one moiety corresponding to Formula (60) or (600) above, therefore, the sum of n and m is preferably 1, 2, 3, 4, or 5; more preferably 1, 2, or 3.

It will be understood that where two or more W₁ and/or W₂ moieties are present on the moiety corresponding to Formula (60) above (i.e., where the sum of n and m is 2, 3, 4, 5, and so on), these moieties will carry similar substituents, each of which may be the same or different as other W₁ and/or W₂ moieties corresponding to the same general structure. For instance and as shown, the W₁ and/or W₂ moieties generally corresponding to Formula (60) carry the R_{333A}, R_{333B}, R_{555A}, R_{555B}, R_{666A}, and R_{666B} substituents, which are defined in connection with the R_{3A}, R_{3B}, R_{33A}, and R_{33B} substituents, the R_{5A}, R_{5B}, R_{55A}, and R_{55B} substituents, and the R_{6A}, R_{6B}, R_{66A}, and R_{66B} substituents, respectively. Additional W₁ and/or W₂ moieties generally corresponding to Formula (60), therefore, may carry the R_{3333A} and R_{3333B} substituents, the R_{5555A} and R_{5555B} substituents, and the R_{6666A} and R_{6666B} substituents, and so on, which are also defined in connection with the R_{3A}, R_{3B}, R_{33A}, and R_{33B} substituents, the R_{5A}, R_{5B}, R_{55A}, and R_{55B} substituents, and the R_{6A}, R_{6B}, R_{66A}, and R_{66B} substituents, respectively, as discussed in detail above.

In one embodiment, the six-membered heterocyclic 1,4-diaza-2-one moieties covalently bonded by a hydrazine bond (i.e., P₁ and P₂) correspond to Formula (50):

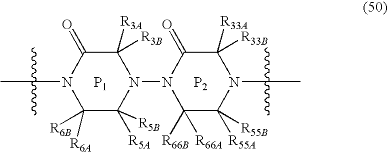

each W₁ and W₂ corresponds to Formula (60):

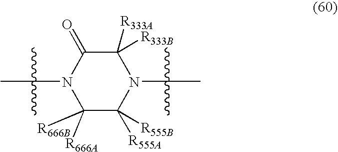

R_{3A}, R_{3B}, R_{33A}, R_{33B}, R_{333A}, and R_{333B} are independently hydrogen, substituted or unsubstituted hydrocarbyl (e.g., substituted or unsubstituted aryl, substituted or unsubstituted alkylenearyl, substituted or unsubstituted alkylaryl, alkanol, alkanoic acid, substituted or unsubstituted alkylene amine, substituted or unsubstituted alkylene amide, substituted or unsubstituted alkyleneguanidine), heterocyclo, alkoxy, ester, thioester, thioether, amino (e.g., substituted or unsubstituted amines such as alkylamine, dialkylamine), or amido (e.g., substituted or unsubstituted amide);

R_{5A}, R_{5B}, R_{55A}, R_{55B}, R_{555A}, and R_{555B} are independently hydrogen, hydrocarbyl, substituted hydrocarbyl or together with the carbon atom to which they are attached form a five- or six-membered ring;

R_{6A} is hydrogen, sulfonyl, or phosphoryl, or together with R_{6B} forms ═O;

R_{6B} is hydrogen or together with R_{6A} forms ═O;

R_{66A} is hydrogen, sulfonyl, or phosphoryl, or together with R_{66B} forms ═O;

R_{66B} is hydrogen or together with R_{66A} forms ═O;

R_{666A} is hydrogen, sulfonyl, or phosphoryl, or together with R_{666B} forms ═O;

R_{666B} is hydrogen or together with R_{666A} forms ═O; and

n is 0 to 10 and m is 0 to 10 provided, however, the sum of n and m is 1 to 10.

In another embodiment, the six-membered heterocyclic 1,4-diaza-2-one moieties covalently bonded by a hydrazine bond (i.e., P₁ and P₂) correspond to Formula (500):

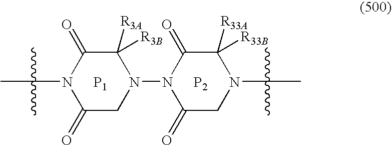

each W₁ and W₂ corresponds to Formula (600):

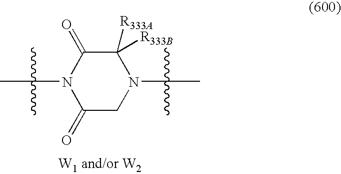

and R_{3A}, R_{3B}, R_{33A}, R_{33B}, R_{333A}, and R_{333B} are independently hydrogen, hydrocarbyl, or substituted hydrocarbyl (such as, for example, unsubstituted C₁-C₈ alkyl (e.g., methyl, ethyl, propyl, butyl, etc.), substituted or unsubstituted aryl (e.g., benzyl, phenyl, napthyl, hydroxybenzyl, etc.) or substituted C₁-C₆ alkyl (e.g., alkanol, alkyl alkylene thioether, alkanoic acid, alkyleneamide, alkyleneamine, alkyleneguanidine, etc.)); and n is 0 to 10 and m is 0 to 10 provided, however, the sum of n and m is 1 to 10. More preferably in this embodiment, R_{3A}, R_{3B}, R_{33A}, R_{33B}, R_{333A}, and R_{333B} are independently hydrogen, alkanoic acid, alkanol, alkyl, substituted alkyl, alkylene amide, alkylene amine, aralkyl, substituted aralkyl, or heteroaralkyl. According to various embodiments, at least one of R_{3A}, R_{3B}, R_{33A}, R_{33B}, R_{333A}, and R_{333B} is other than hydrogen.

In either of the two preceeding embodiments, for example, the sum of n and m is preferably 1, 2, 3, 4, or 5; more preferably 1, 2, or 3.

The Z₁ and Z₂ Moieties

In addition to the —P₁—, —P₂—, —(W₁)ₙ—, and —(W₂)ₘ— substituents, the formula also carries the substituents Z₁ and Z₂. In general, the substituents at Z₁ and Z₂ are groups which are utilized in the preparation of the compositions, or are groups which can affect certain interactions (e.g., electrostatic, hydrophobic, and/or mimetic interactions) between the composition and a target protein, affect solubility or other pharmacokinetic properties, and/or affect the ability of the composition to penetrate cellular membranes. Thus, the compositions may have a particular substituent at Z₁ and/or Z₂ during synthesis of the composition which may be subsequently cleaved and replaced with another (e.g., different) Z₁ and/or Z₂ substituent during synthesis or once synthesis is complete or in preparation of the composition for use, e.g., in binding assays, as analytical agents, as agents to be used to isolate or purify proteins, as intermediates in the synthesis of further peptido- and/or proteomimetic agents, or as active agents in pharmaceutical compositions.

Although Z₁ and Z₂ may be independently hydrogen, hydrocarbyl (e.g., alkyl, alkenyl, alkynyl, or aryl), substituted hydrocarbyl (e.g., substituted alkyl, alkenyl, alkynyl, or aryl), heterocyclo, acyl, alkoxy, amino, protected amino, sulfonyl, phosphoryl, or a linker moiety attached to a solid support, in certain embodiments Z₁ and Z₂ are independently hydrogen, hydrocarbyl, substituted hydrocarbyl, heterocyclo, acyl, amino, protected amino, sulfonyl, or phosphoryl. In certain other embodiments, Z₁ is a linker moiety attached to a solid support, and Z₂ is hydrogen, hydrocarbyl, substituted hydrocarbyl, heterocyclo, acyl, amino, protected amino, sulfonyl, or phosphoryl; thus, for example, Z₁ may be a linker moiety attached to a solid support, and Z₂ may be amino or protected amino.

As noted above, the Z₁ and/or Z₂ substituents may be selected from groups which can affect solubility or other pharmacokinetic properties, and/or affect the ability of the composition to penetrate cellular membranes. Thus, for example, the Z₁ and Z₂ moieties can consist of polar groups (e.g., alkoxy, alkanol, carboxyl, carboxylate, amino, amido, guanidinium, amidinium, and the like), non-polar groups (e.g., hydrocarbyl such as alkyl (e.g., methyl, ethyl, propyl, butyl, pentyl, hexyl, octyl, decyl, benzyl, and the like), groups which are capable of salt-bridge interactions (e.g., carboxylates which can interact with positively-charged residues on the target protein), and the like.

Stereoisomers

With regard to stereoisomers, it should be understood that a solid line designation for the bonds in the compositions corresponding to Formulae (50), (500), (60), (600) (and others herein) for attachment of an R group (e.g., R_{3A}, R_{3B}, R_{33A}, R_{33B}, R_{333A}, R_{333B}, and so on) to a chiral carbon atom of the compound indicates that these groups may lie either below or above the plane of the page (i.e., [symbol] R or [symbol] R). All isomeric forms of the compositions disclosed herein are contemplated, including racemates, racemic mixtures, and individual enantiomers or diastereomers. In various embodiments, the compositions of the present invention have a stereoconformation intended to mimic the helix of L-form amino acids, D-form amino acids, or mixtures thereof, such as the alpha-helix. Thus, in various embodiments the six-membered heterocyclic 1,4-diaza-2-one moieties covalently bonded by a hydrazine bond (i.e., P₁ and P₂) correspond to Formulae (5000):

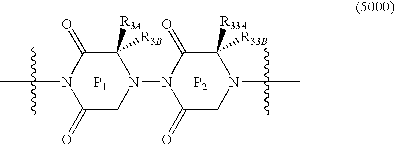

wherein R_{3A}, R_{3B}, R_{33A}, and R_{33B} are as defined in connection with Formula (50). Thus, for example, when the six-membered heterocyclic 1,4-diaza-2-one moieties covalently bonded by a hydrazine bond (i.e., P₁ and P₂) corresponds to Formula (5000), R_{3A}, R_{3B}, R_{33A}, and R_{33B} may be independently hydrogen, hydrocarbyl, or substituted hydrocarbyl. In a particular embodiment in which the six-membered heterocyclic 1,4-diaza-2-one moieties covalently bonded by a hydrazine bond (i.e., P₁ and P₂) corresponds to Formula (5000), R_{3A}, R_{3B}, R_{33A}, and R_{33B} are independently hydrogen, alkanoic acid, alkanol, alkyl, substituted alkyl, alkylene amide, alkylene amine, aralkyl, substituted aralkyl, or heteroaralkyl. According to various embodiments in which the six-membered heterocyclic 1,4-diaza-2-one moieties covalently bonded by a hydrazine bond (i.e., P₁ and P₂) corresponds to Formula (5000), at least one of R_{3A}, R_{3B}, R_{33A}, and R_{33B} is o than hydrogen.

Similarly, in various embodiments the W₁ and/or W₂ moieties correspond to Formula (6000):

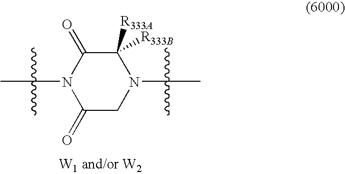

wherein R_{333A} and R_{333B} are as defined in connection with Formula (50). Thus, for example, when the six-membered heterocyclic rings (i.e., W₁ and/or W₂) correspond to Formula (6000), R_{333A} and R_{333B} may be independently hydrogen, hydrocarbyl, or substituted hydrocarbyl. In a particular embodiment in which the six-membered rings (i.e., W₁ and/or W₂) correspond to Formula (6000), R_{333A} and R_{333B} are independently hydrogen, alkanoic acid, alkanol, alkyl, substituted alkyl, alkylene amide, alkylene amine, aralkyl, substituted aralkyl, or heteroaralkyl. According to various embodiments in which the six-membered rings (i.e., W₁ and/or W₂) correspond to Formula (6000), at least one of R_{333A} and R_{333B} is other than hydrogen.

Chemical Synthesis

Other aspects of the present invention are directed to processes for the preparation of compositions containing two, or more, six-membered heterocyclic 1,4-diaza-2-one moieties covalently bonded by a hydrazine bond, and intermediates useful in preparing such compositions, such as single six-membered heterocyclic 1,4-diaza-2-one moieties. The compositions described herein (e.g., compositions having the formula Z₁—(W₁)ⁿ—P₁-P₂—(W₂)ₘ—Z₂, wherein P₁, P₂, W₁, W₂, Z₁, Z₂, n and m are defined above (e.g., in connection with Formulae (50), (500), (5000), (60), (600), and (6000))) are prepared using the general and specific synthetic methods set forth below.

Reaction Scheme 1 illustrates the general synthetic method for preparing six-membered heterocyclic 1,4-diaza-2-one intermediates. In Reaction Scheme 1, L₁ and L₂ are independently hydrogen, hydrocarbyl, or substituted hydrocarbyl, Pr is an amino protecting group, and R_{3A}, R_{3B}, R_{5A}, R_{5B}, R_{6A}, and R_{6B} are as defined in connection with Formula (50). The intermediates produced according to the processes described in Reaction Scheme 1 may be subsequently coupled with other similarly prepared six-membered heterocyclic 1,4-diaza-2-one intermediates as described herein to form two, three, or more, six-membered heterocyclic 1,4-diaza-2-one moieties covalently bonded by hydrazine bonds.

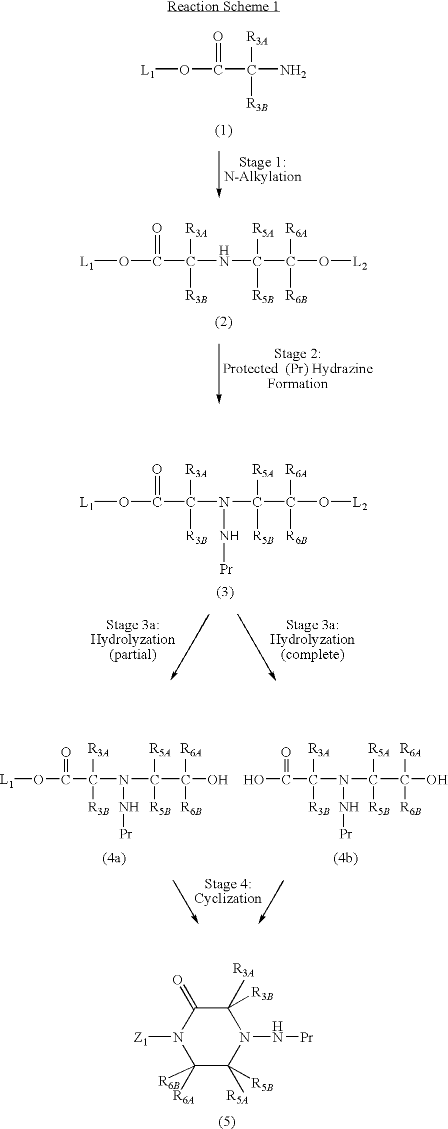

N-Alkylation

Stage 1 of Reaction Scheme 1 illustrates the N-alkylation of an amino acid or ester thereof; that is, the attachment of a substituted alkyl moiety to the primary amine of the amino acid or ester thereof. As shown, the amino acid or ester corresponds to Formula (1), wherein L₁ is hydrogen, hydrocarbyl, or substituted hydrocarbyl, and R_{3A} and R_{3B} are as defined in connection with Formula (50). Typically, one of R_{3A} and R_{3B} will be hydrogen and the other of R_{3A} and R_{3B} will be a group encompassing an amino acid side chain moiety as described above.

Generally speaking, any naturally occurring or non-naturally occurring amino acid or ester corresponding to Formula (1) may be utilized as the starting material. The selection of a particular amino acid or ester generally depends on the desired substituents attached to the central (α-) carbon atom (i.e., R_{3A} and R_{3B}) in addition to the amino moiety and the carboxyl or carboxy ester moiety. Thousands of amino acids and/or amino acid esters are commercially available from such vendors as Sigma Chemical Co. (St. Louis, Mo.) and Bachem Bioscience Inc. (King of Prussia, Pa.).

Where the starting material is an amino acid, the L₁ moiety of amino acid or ester (1) will be hydrogen, and where the starting material is an amino acid ester, the L₁ moiety of amino acid or ester (1) will be hydrocarbyl or substituted hydrocarbyl. Typically, the starting material is an amino acid alkyl or aryl ester (i.e., L₁ is alkyl (e.g., methyl, ethyl, propyl, etc.) or aryl (e.g., phenyl, benzyl, etc.). Amino acid alkyl or aryl esters may be prepared according to conventional methods (see Example 1), or are commercially available from a variety of sources.

Although the amino acid or ester (1) is shown in the unionized form, it will be understood that the amino acid or ester (1) may also be utilized in the anionic, or conjugate base, form, in combination with a cation. Suitable cations include alkali metal ions, such as sodium and potassium ions, alkaline earth metal ions, such as calcium and magnesium ions, and unsubstituted and substituted (primary, secondary, tertiary, and quaternary) ammonium ions. Suitable cations also include transition metal ions such as manganese, copper, nickel, iron, cobalt, and zinc. The basic amino group can also be protonated in combination with a counterion such as hydroxide, a halogen (e.g., chloride, bromide, or iodide), acetate, formate, citrate, ascorbate, sulfate, or phosphate.

In general, any alkylating agent and/or alkylation method may be utilized in the Stage 1 N-alkylation reaction, provided that the substituents carried by the alkylating agent (i.e., R_{5A}, R_{5B}, R_{6A}, R_{6B}, and —O-L₂) are positioned such that the resulting secondary amine corresponds to Formula (2), wherein L₂ is hydrogen, hydrocarbyl, or substituted hydrocarbyl. Typically, at least one of L₁ (on the amino acid or ester (1)) and L₂ (on the alkylating agent) is not hydrogen. Suitable methods for the N-alkylation of the amino acid or ester (1) to form the secondary amine (2) include, for instance, conventional amine alkylation or reductive amination procedures.

Regardless of the particular alkylation method employed, the N-alkylation reaction is typically carried out in the presence of a base. The base included in the reaction mixture may be an organic (e.g., an amine base) or inorganic base. Preferably, the base is an amine base. Suitable amine bases include, for example, triethylamine (TEA); tributylamine; N,N-dicyclohexylmethylamine; diisopropylamine; N,N-diisopropylmethylamine; N,N-diisopropylethylamine (DIEA); N,N-diisopropyl-2-ethylbutylamine; N,N-diisopropyl-3-pentylamine; N,N,N′,N′-tetramethyl-1,8-naphthalenediamine; tris(trimethylsilyl)amine; N,N-diethylaniline; N,N-dimethylaniline; 1,1,3,3-tetramethylguanidine; 2-tert-butyl-1,1,3,3-tetramethyl-guanidine; imidazole and imidazole derivatives; 2,6-lutidine; 1,2,2,6,6-pentamethylpiperidine (PMP); 2,2,6,6-tetramethylpiperidine (TMP); pyridine; N,N-4-dimethylaminopyridine (DMAP); 2,4,6-trimethylpyridine; 2,6-di-tert-butyl-4-methylpyridine; 2,4,6-tri-tert-butylpyridine; 1,8-diazabicyclo[5.4.0]undec-7-ene (DBU); 1,5-diazabicyclo[4.3.0]non-5-ene (DBN); 1,4-diazabicyclo(2.2.2)octane (TED); 7-methyl-1,5,7-triazabicyclo(4.4.0)dec-5-ene (MTBD); 3,3,6,9,9-pentamethyl-2,10-diazabicyclo-(4.4.0)dec-1-ene (PMDBD); 1,5,7-triazabicylco(4.4.0)dec-5-ene; quinuclidine; and the like, and mixtures thereof. Among certain of the preferred amine bases are triethylamine (TEA) and N,N-diisopropylethylamine (DIEA).

To assist in the formation of a relatively homogeneous reaction mixture (e.g., to solubilize the amino acid or ester (1)), the N-alkylation reaction is typically conducted in an organic solvent. Typically, the amino acid or ester (1) is combined with the organic solvent in the reaction vessel prior to the addition of the alkylating agent, the base, and/or any other reagents utilized in the N-alkylation reaction. Alternatively, however, the organic solvent, the alkylating agent, and/or the base may be combined and thereafter added to the reaction vessel containing the amino acid or ester (1). Exemplary organic solvents that may be used to solubilize the amino acid or ester (1) prior to or during the N-alkylation reaction include, but are not limited to, aprotic dipolar solvents (such as acetone, acetonitrile, dimethylformamide, dimethylacetamide, dimethylsulfoxide, 1-methyl-2-pyrrolidinone, and the like), alcohols (such as methanol, ethanol, tert-butanol, isopropanol, and the like), combinations thereof, and the like.

Depending on the particular amino acid or ester (1), alkylating agent, and/or N-alkylation method, the reaction temperatures and times may vary. Typically, the alkylation reaction is carried out at room temperature (e.g., 20° C. to 25° C.) or cooler. Performing the alkylation at warmer temperatures generally tends to increase side product formation (e.g., overalkylation). Similarly, longer reaction times also generally tend to increase side product formation. Reaction times can range from 1 hour to 24 hours, or longer; e.g., from about 1 hour to about 12 hours.

Protected Hydrazine Formation

Stage 2 of Reaction Scheme 1 illustrates the formation of a protected hydrazine (3). In general, any method of introducing a protected amino moiety to the secondary amine (2) to form the protected hydrazine (3) may be employed, provided that the method does not disturb any of the other substituents on the secondary amine (2) (e.g., L₁, L₂, R_{3A}, R_{3B}, R_{5A}, R_{5B}, R_{6A,} and/or R_{6B}). Several procedures for the formation of substituted hydrazines have been reported, each of which and others may be employed. For instance, Colton et al. reported the reaction of alkyl ureas with hypochlorite under basic conditions (see J. Am. Chem. Soc. 76, 2572-4 (1954); Sheppard et al. reported the reaction of a N-monohalomono-substituted urea with an alkaline material in the presence of a reactant affording an active hydrogen in an organic or aqueous reaction medium (see U.S. Pat. No. 3,746,760); Ragnarson reported direct substitution of hydrazine with triphenylbismuthane in the presence of copper acetate (see Chem. Soc. Rev. 30, 205-13 (2001)); Oguz et al. reported the nitrosation of a secondary amine, followed by its selective reduction and protection (see Tetrahedron Letters 43(15), 2873-75 (2002); and Vidal et al. (J. Org. Chem. 58(18), 4791-93 (1993)) reported electrophilic amination methods using N-protected oxaziridines.

Regardless of the particular method utilized to introduce the protected amino moiety to the secondary amine (2), standard amino protecting groups (Pr) may be employed including, for example, benzyl, benzoyl, carbobenzyloxy (Cbz), tert-butoxycarbonyl (Boc or t-Boc), allyloxycarbonyl, fluorenylmethoxycarbonyl (Fmoc), and the like. Typically, the amino protecting group (Pr) is Boc.

Hydrolysis

Following the formation of the protected hydrazine (3) in Stage 2, the compound is partially or completely hydrolyzed in Stage 3a or Stage 3b to form a carboxylic acid (4), shown in Reaction Scheme 1 as monoester (4a) and diacid (4b). Hydrolyzation generally involves treating the protected hydrazine (3) with a hydrolyzing agent that will remove one (i.e., in the case of partial hydrolysis) or both (i.e., in the case of complete hydrolysis) of the hydroxy protecting groups present as the L₁ and L₂ moieties (e.g., hydrocarbyl or substituted hydrocarbyl); thus forming either the monoester (4a) or the diacid (4b) in the case of partial and complete hydrolysis, respectively, as shown in Reaction Scheme 1. Depending on the definitions of L₁ and L₂, therefore, it may not be necessary to perform the hydrolyzation step. For example, if an amino acid is utilized as the starting material instead of an amino acid ester (i.e., L₁ is hydrogen) and if the L₂ moiety of the particular alkylating agent employed is hydrogen, then the protected hydrazine (3) will already possess the desired carboxyl moieties that would result, for example, in the case of complete hydrolysis. As noted above, however, at least one of L₁ and L₂ is typically other than hydrogen. By way of another example, if an amino acid is utilized as the starting material instead of an amino acid ester (i.e., L₁ is hydrogen) and the L₂ substituent is other than hydrogen, then the protected hydrazine (3) will already posses the desired carboxyl moiety that would result, for example, in the case of partial hydrolysis.

The hydrolyzing agent may be any relatively mild hydrolyzing agent that will not disturb the amino protecting group (Pr) and/or the other various substituents on the protected hydrazine (3) (e.g., R_{3A}, R_{3B}, R_{5A}, R_{5B}, R_{6A}, and/or R_{6B}). Suitable hydrolyzing agents include organic and inorganic acids, bases, and alcohols. For example, the hydrolyzing agent may be a base such as sodium hydroxide, potassium hydroxide, lithium hydroxide, cesium hydroxide, sodium carbonate, tetramethylammonium hydroxide, and the like. Alternatively, the hydrolyzing agent may be a reducing agent (e.g., for chemical or catalytic reduction). Representative reducing agents for use in chemical reduction include hydrides (e.g., hydrogen iodide, hydrogen sulfide, lithium aluminum hydride, sodium borohydride, sodium cyanoborohydride, and the like), or combinations of a metal (e.g., tin, zinc, or iron) or a metal compound (e.g., chromium chloride, chromium acetate, and the like) with an organic or inorganic acid (e.g., formic acid, acetic acid, propionic acid, trifluoroacetic acid, p-toluenesulfonic acid, hydrochloric acid, and the like), samarium iodide, and others. Representative reducing agents for use in catalytic reduction methods include commonly used catalysts such as, for example, platinum catalysts (e.g., platinum black, colloidal platinum, platinum oxide, platinum plate, platinum sponge, platinum wire, and the like), palladium catalysts (e.g., palladium black, palladium on barium carbonate, palladium on barium sulfate, colloidal palladium, palladium on carbon, palladium hydroxide on carbon, palladium oxide, palladium sponge, and the like), nickel catalysts (e.g., nickel oxide, Raney nickel, reduced nickel, and the like), cobalt catalysts (e.g., Raney cobalt, reduced cobalt, and the like), iron catalysts (e.g., Raney iron, reduced iron, Ullmann iron, and the like), and others. Regardless of the particular hydrolyzing agent employed, the hydrolyzation reaction is typically carried out in the presence of a solvent. Suitable solvents include inert organic solvents such as, for example, alcohols (such as aqueous methanol, ethanol, tert-butanol, and isopropanol), and the like), aromatic hydrocarbons (e.g., benzene, tetrahydronaphthalene, toluene, xylene, and the like), halogenated aromatic hydrocarbons (e.g., bromobenzene, chlorobenzene and o-dichlorobenzene, fluorobenzene, hexafluorobenzene, and the like), ethers (e.g. anisole, butyl ether, 1,2-diethoxyethane, diethylene glycol dimethyl ether, diethylene glycol diethyl ether, diethyl ether, 1,2-dimethoxyethane, dimethoxymethane, 1,4-dioxane, diphenyl ether, ethylene glycol dimethyl ether, ethylene glycol diethyl ether, isopropyl ether, methyl-t-butyl ether, propyl ether, tetrahydrofuran, and the like), nitriles (e.g., acetonitrile or benzonitrile), amides (e.g., hexamethylphosphoramide) and thioamides (e.g., dimethylthioformamide, dimethylthioacetamide or tetraethylsulfamide), and mixtures thereof

The reaction temperature for the hydrolysis reaction is typically from about 0° C. to about 35° C.; preferably room temperature (e.g., from about 20° C. to about 25° C. Reaction times can range from about 1 hour to about 24 hours; e.g., from about 1 hour to about 12 hours. It will be understood that the particular hydrolyzating agents and hydrolyzation reaction conditions may be varied depending on whether the monoester (4a) or the diacid (4b) is desired (i.e., in the case of partial or complete hydrolysis, respectively), and the particular substituents present at L₁ and L₂ and their relative ease of removal. For instance, where both L₁ and L₂ are other than hydrogen, the hydrolyzation reaction conditions and hydrolyzation agent(s) can be selected and controlled to selectively remove only the L₁ substituent (i.e., where partial hydrolysis is desired) or both the L₁ and the L₂ substituents (i.e., where complete hydrolysis is desired).

Cyclization

Stage 4 of Reaction Scheme 1 illustrates the cyclization of the monoester (4a) or diacid (4b) (collectively refered to as carboxylic acid (4)) to form the six-membered heterocyclic 1,4-diaza-2-one (5). In general, the cyclization reaction involves the treatment of the carboxylic acid (4) with an activating agent to activate the carboxylic acid (4) towards amide formation, closing the ring to form the six-membered heterocyclic 1,4-diaza-2-one (5). Conventional cyclization methods may be employed in the Stage 4 cyclization reaction including, for example, those performed in combination with conventional solution or solid phase synthesis methods, or a combination of solution and solid phase synthesis methods.

A variety of conventional activating agents may be employed in this step for the activation of carboxylic acid (4) to form the cyclized six-membered heterocyclic 1,4-diaza-2-one (5). For instance, suitable coupling agents for use in the cyclization reaction include, but are not limited to aromatic oximes such as O-(7-azabenzotriazol-1-yl)-tris(dimethylamino)phosphonium (AOP), 1-benzotriazol-1-yloxy-bis(pyrrolidino)uronium hexafluorophosphate (BBC), 5-(1H-benzotriazol-1-yloxy)-3,4-dihydro-1-methyl 2H-pyrrolium hexachloroanitimonate (BDMP), benzotriazol-1-yl diethyl phosphate (BDP), 2-(benzotriazol-1-yl)oxy-1,3-dimethyl-imidazolinium hexafluorophosphate (BOI), benzotriazol-1-yloxy-N,N-dimethylmethaniminium hexachloroantimonate (BOMI), benzotriazolyl-1-oxy-tris(dimethylamino)phophonium hexafluorophosphate (BOP), bis(2-oxo-3-oxazolidinyl)phosphinic chloride (BOP-Cl), bromotris(dimethylamino)phophonium hexafluorophosphate (BroP), carbonyldiimidazole (CDI), 2-chloro-1,3-dimethylimidazolidinium hexafluorophosphate (CIP), 1,3-dicyclohexylcarbodiimide (DCC), 3-(diethoxyphosphoryloxy)-1,2,3-benzotriazin-4(3H)-one (DEPBT), diisopropylcarbodiimide (DIC), diphenylphosphinic chloride (Dpp-Cl), 1-ethyl-3-(3′-dimethylaminopropyl)carbodiimide hydrochloride (EDC), 2-ethoxy-1-ethoxycarbonyl-1,2-dihydroquinoline (EEDQ), pentafluorophenyl diphenylphosphinate (FDPP), O-(7-azabenzotriazol-1-yl)-1,1,3,3-bis(tetramethylene)uranium hexafluorophosphate (HAPyU), O-(7-azabenzotriazol-1-yl)-1,1,3,3-tetramethyluranium hexafluorophosphate (HATU), O-(benzotriazol-1-yl)-1,1,3,3-tetramethyluranium hexafluorophosphate (HBTU), 1-hydroxy-7-azabenzotriazole (HOAt), hydroxybenzotriazole (HOBt), 3-hydroxy-3,4-dihydro-4-oxo-1,2,3-benzotriazine (HODhbt), hydroxysuccinimide (HOSu), S-(1-oxido-2-pyridinyl)-1,1,3,3-tetramethylthiouroniumn hexafluorophosphate (HOTT), 2,4,6-mesitylenesulfonyl-3-nitro-1,2,4-triazolide (MSNT), N-ethyl-5-phenylisoxazolium-3′-sulfonate (NEPIS), 7-azobenzotriazolyoxytris(pyrrolidino)phosphonium hexafluorophosphate (PyAOP), benzotriazolyl-1-oxy-tripyrrolidinophosphonium hexafluorophosphate (PyBOP), bromotripyrrolidinophosphoniurn hexafluorophosphate (PyBroP), chlorotris(pyrrolydino)phophonium hexafluorophosphate (PyCloP), O-(7-azabenzotriazol-1-yl)-1,1,3,3-bis(pentamethylene)uranium tetrafluoroborate (TAPipU), O-(benzotriazol-1-yl)-1,1,3,3-tetramethyluranium tetrafluoroborate (TBTU), 2-(3,4-dihydro-4-oxo-1,2,3-benzotriazin-3-yl)-1,1,3,3-tetramethyluronium tetrafluoroborate (TDBTU), 2,5-diphenyl-2,3-dihydro-3-oxo-4-hydroxythiophene dioxide (TDO), O-[(5-norbornene-2,3-dicarboximido)-1,1,3,3-tetramethyluronium tetrafluoroborate (TNTU), S-(1-oxido-2-pyridinyl)-1,1,3,3-tetramethylthiouronium tetrafluoroborate (TOTT), O-[(cyano(ethoxycarbonyl)methylene)amino]-1,1,3.3-tetramethyluronium tetrafluoroborate (TOTU), O-(1,2-dihydro-2-oxo-1-pyridyl-1,1,3,3-tetramethyluronium tetrafluoroborate (TPTU), O-(N-succinimidyl)-1,1,3,3-tetramethyluronium tetrafluoroborate (TSTU), and the like. Solvents for use with such activating agents in the cyclization reaction include aprotic dipolar solvents such as acetone, acetonitrile, chloroform, dichloromethane, dimethylformamide, N-methyl-pyrrolidinone, tetrahydrofuran, and the like.

Alternatively, activation of the carboxylic acid (4) to form the cyclized six-membered heterocyclic 1,4-diaza-2-one (5) can be carried out using an activating agent including a non-nucleophilic base. Representative non-nucleophilic organic bases include, but are not limited to, triethylamine, tripropylamine, N,N-diisopropylethylamine, N-methylmorpholine, N-ethylmorpholine, and the like. Examples of suitable solvents for use in combination with such activating agents include, but are not limited to, acetonitrile, chloroform, dichloromethane, 1,2-dimethoxyethane, diethyl ether, diisopropyl ether, and tetrahydrofuran.

By way of another alternative, the activation of the carboxylic acid (4) to form the cyclized six-membered heterocyclic 1,4-diaza-2-one (5) can be carried out by the formation of a mixed anhydride using an activating agent including an alkyl chloroformate or alkanoyl chloride in the presence of a non-nucleophilic base. Suitable alkyl chloroformate or alkanoyl chlorides include, for instance, methyl chloroformate, ethyl chloroformate, propyl chloroformate, butyl chloroformate, isobutyl chloroformate, pivaloyl chloride, adamantine carboxyl chloride, and the like. Representative non-nucleophilic organic bases and solvents for use in these cyclization reactions are described above.

Other suitable condensing/activating agents that may be used in the activation of the carboxylic acid (4) to form the cyclized six-membered heterocyclic 1,4-diaza-2-one (5) include, for example, acetic acid in 4-(Dimethylamino)pyridine (DMAP), mercuric trifluoroacetate (Hg(OOCCF₃)₂), and the like. Still other suitable agents for the cyclization reaction include bases such as potassium hydride, sodium hydride, potassium amide, sodium amide, potassium diisopropylamide, sodium diisopropylamide, lithium diisopropylamide, potassium hexamethylsilazide, sodium hexamethylsilazide, lithium hexamethylsilazide, potassium tert-butoxide, sodium tert-butoxide, lithium tert-butoxide, and strong neutral bases such as DBU or DBN, or others, which are capable of deprotonating an amide (as discussed below).

The various reaction conditions for the cyclization reaction using the above activating agents are generally known. Typically, the reaction temperature for the cyclization reaction is from about 25° C. to about 180° C.; e.g., from about 100° C. to about 150° C. Reaction times can range from about 1 hour to about 72 hours; e.g., from about 1 hour to about 48 hours.

Reaction Scheme 2a illustrates the preparation of six-membered heterocyclic 1,4-diaza-2-one (5) intermediates according to several different synthetic pathways noted above.

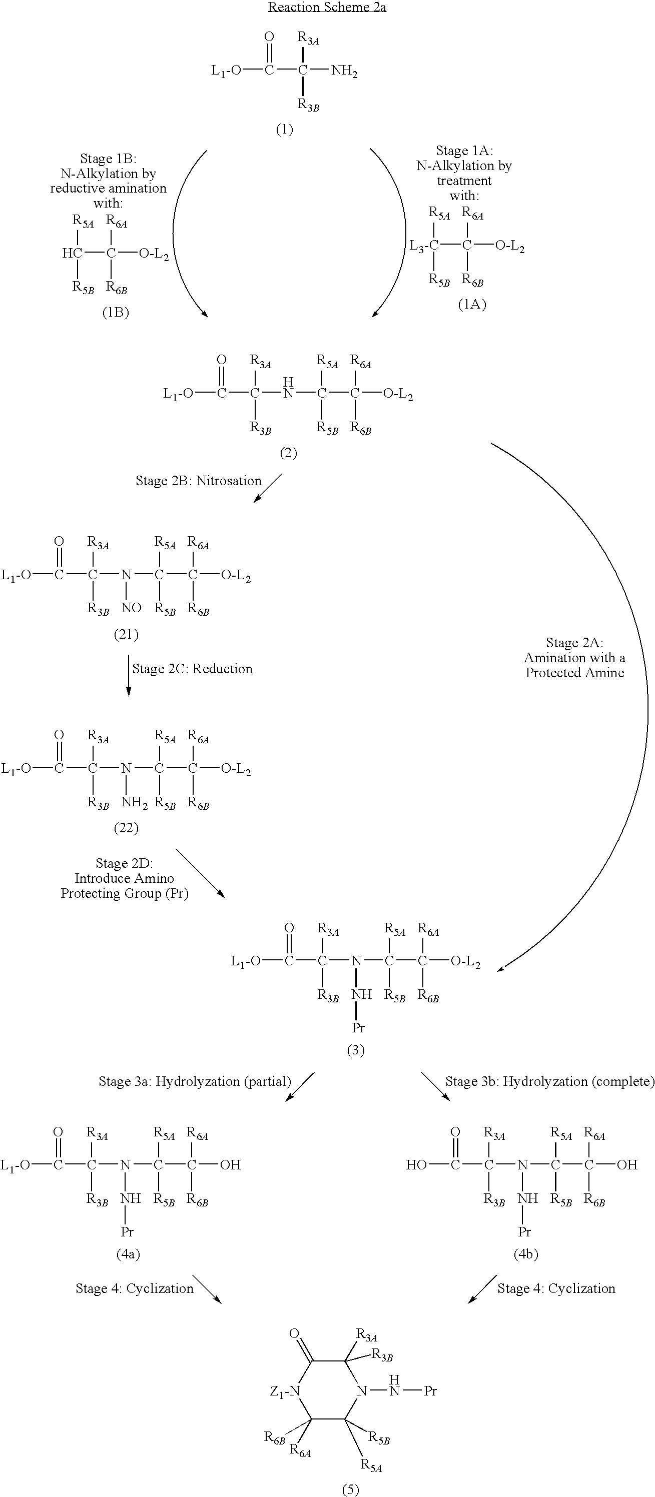

As illustrated in Reaction Scheme 2a, the secondary amine (2) may be prepared by two different synthetic pathways, shown as Stage 1A and Stage 1B. According to the Stage 1A reaction, the alkylating agent corresponds to halo-alkyl-acetate (1A) wherein L₃ is halo, L₂ is described above, and R_{3A}, R_{3B}, R_{5A}, R_{5B}, R_{6A}, and R_{6B} are as defined in connection with Formula (50). Suitable alkylating agents falling within the structure and substituent definitions of the above formula include, for example, methyl bromoacetate, ethyl bromoacetate, and the like. Suitable bases and solvents for this N-alkylation reaction are described in detail above.

Alternatively, reductive amination may be employed in the formation of the secondary amine (2), as shown in the Stage 1B reaction. According to conventional reductive amination methods, the alkylating agent preferably includes at least one carbonyl group (e.g., R_{6A} and R_{6B} together form ═O and/or R_{5A} and R_{5B} together form ═O; preferably R_{5A} and R_{5B} together form ═O). Suitable alkylating agents for use in reductive amination of the amino acid or ester thereof include, for example, glyoxylic acid, ethylglyoxylate, benzylglyoxylate, and the like. In this reaction, the carbonyl group preferably present on the alkylating agent reacts with the amino moiety of the amino acid or ester thereof, forming an imine that is concurrently or subsequently reduced with a reducing agent such as sodium borohydride (NaBH₄), sodium cyanoborohydride (NaBH₃CN), sodium triacetoxyborohydride (NaBH(OCOCH₃)), and the like, to form the secondary amine (2).

Next, the protected amine moiety is introduced to the secondary amine (2) according to various synthetic pathways. For example, in Stage 2A, electrophilic amination is utilized to directly introduce a protected amino group to the secondary amine (2) in a single step, forming the protected hydrazine (3). For example, Vidal et al. (J. Org. Chem. 58(18), 4791-93 (1993)) described the use of certain oxaziridine compounds for introducing protected amino groups to N- and C-nucleophiles. In general, conditions for the Stage 2A reaction are similar to those described by Vidal et al. In a particular embodiment, an oxaziridine including a protected amino moiety is employed; thus, for example, the oxaziridine may be tert-butyl-3-(trichloromethyl)-1,2-oxaziridine-2-carboxylate. Alternatively, however, other suitable N-protected oxaziridines may be employed. Generally, a slight to moderate excess of the oxaziridine is employed, for example, greater than 1 equivalent; typically, 2 to 3 equivalents, or more, are employed. Regardless of the particular oxaziridine utilized to directly introduce the protected amino moiety to the secondary amine (2), standard amino protecting groups may be employed including, for example, benzyl, benzoyl, carbobenzyloxy (Cbz), tert-butoxycarbonyl (Boc or t-Boc), allyloxycarbonyl, fluorenylmethoxycarbonyl (Fmoc), and the like. Typically, the amino protecting group (Pr) is Boc.

Alternatively, as shown in Stages 2B, 2C, and 2D of Reaction Scheme 2a, the protected amino moiety may be introduced to the secondary amine (2) in a series of steps. According to this pathway, the secondary amine (2) is first nitrosated to attach an —NO moiety to the secondary amine moiety by treatment with a nitrosating agent. Any NO⁺ source may be utilized as the nitrosating agent. Suitable nitrosating agents include, for example, alkyl nitrites (i.e., R—ONO, where R is alkyl) such as ethyl nitrite, butyl nitrite, isobutyl nitrite, or tert-butyl nitrite; alkali metal nitrites such as sodium nitrite; nitrosyl sulfuric acid or salts thereof; nitrogen dioxide (NO₂); nitrogen trioxide (NO₃); nitrogen peroxide (NO₄); nitrous oxide (NO) associated with an oxidizing agent such as nitric acid, nitrogen dioxide, or oxygen; mixtures thereof; and the like.

After treatment of the secondary amine (2) with a nitrosating agent, the resulting nitrosamine (21) is treated with a reducing agent (shown as Stage 2C) to selectively reduce the —NO moiety to the primary amine (i.e., —NH₂). A wide variety of reducing agents may be employed in this step including, for example, catalytic hydrogenation (e.g., H₂/PtO), Fe/H⁺, Sn/H⁺, Zn/H⁺, samarium iodide, Raney nickel, and the like, and others noted above in connection with the hydrolyzing agent.

Following formation of the unprotected hydrazine (22) by way of selective reduction of the nitrosamine (21), the —NH₂ moiety may be protected with an amino protecting group according to conventional methods. Thus, the unprotected hydrazine (22) may be protected with standard amino protecting groups including, for example, benzyl, benzoyl, carbobenzyloxy (Cbz), tert-butoxycarbonyl (Boc or t-Boc), allyloxycarbonyl, fluorenylmethoxycarbonyl (Fmoc), and the like. Typically, the amino protecting group (Pr) is Boc.

Regardless of the particular method for introducing a protected amino moiety to the secondary amine (2), these protected hydrazine-formation reaction(s) is/are preferably carried out under relatively cool temperatures in order to prevent decomposition of the product. Thus, for example, introduction of the protected amino group is typically carried out at a reaction temperature of from about −100° C. to room temperature; e.g., from about −80° C. to about −20° C. The hydrazine formation reaction is also preferably conducted under relatively anhydrous conditions. Reaction times can vary, but are typically less than about 12 hours; thus, for example, reaction times can range from about 1 hour to about 6 hours, or from about 2 hours to about 5 hours.

Following formation of the protected hydrazine (3) according Stages 1A→2A, Stages 1A→2B→2C→2D, Stages 2A→2B→2C→2D, or Stages 1B→2A, the protected hydrazine may be hydrolyzed and cyclized in Stages 3 and 4 as described above in connection with Reaction Scheme 1 (e.g., conventional cyclization techniques described above and otherwise known to those of skill in the art may be employed). Reaction Scheme 2b shows two exemplary cyclization pathways from the monoester (4a) and the diacid (4b) formed after partial and complete hydrolysis of the protected hydrazine (3), respectively.

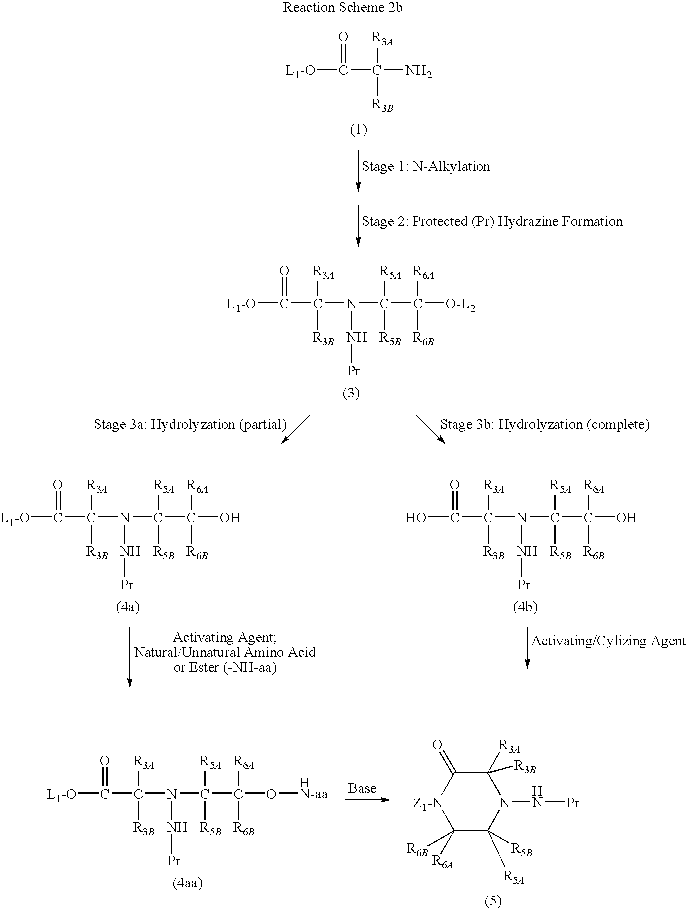

Stages 1, 2, 3 a, and 3b in Reaction Scheme 2b refer to the various synthetic stages described in connection with Reaction Schemes 1 and 2a (i.e,. N-alkylation, protected hydrazine formation, and hydrolysis), with R_{3A}, R_{3B}, R_{5A}, R_{5B}, R_{6A}, R_{6B}, L₁, L₂, and Pr defined in connection with Reaction Schemes 1 and 2a.

As shown in Reaction Scheme 2b, where the diacid (4b) is formed upon (complete) hydrolysis of the protected hydrazine (3) in stage 3b, for example, the resulting hydrolyzed compound can be cyclized using an activating/cyclizing agent to form the cyclized six-membered heterocyclic 1,4-diaza-2-one (5). A variety of aromatic oximes and carbodiimides such as those described above can be employed as the activating/cyclizing agent according to conventional processes (e.g., DCC).

Alternatively, where the monoester (4a) is formed following (partial) hydrolysis of the protected hydrazine (3), the monoester (4a) can be treated with a natural or unnatural amino acid or ester thereof (shown in Reaction Scheme 2a as —NH-aa) in the presence of an activating agent to form a hydrazine amide intermediate (4aa), which may then be treated with a deprotonating base (e.g., sodium hydride) to form the cyclized six-membered heterocyclic 1,4-diaza-2-one (5) (wherein the Z₁ moiety present on the cyclized six-membered heterocyclic 1,4-diaza-2-one (5) represents the natural or unnatural amino acid moiety (i.e., -aa)). Suitable activating agents for coupling the amino acid or ester thereof to the monoester (4a) are described in detail above (e.g., EDC) and are generally known in the field of peptide synthesis. Following attachment of the amino acid (or ester thereof) moiety, cyclization can be carried out by treating the resulting hydrazine amide intermediate (4aa) with a base. A variety of bases may be employed for cyclization of the hydrazine amide (4aa), including, for example, potassium hydride, sodium hydride, potassium amide, sodium amide, potassium diisopropylamide, sodium diisopropylamide, lithium diisopropylamide, potassium hexamethylsilazide, sodium hexamethylsilazide, lithium hexamethylsilazide, potassium tert-butoxide, sodium tert-butoxide, lithium tert-butoxide, and strong neutral bases such as DBU or DBN, or others, which can deprotonate the amide intermediate.

According to the latter approach (i.e., formation of the hydrazine amide (4aa), followed by cyclization of the same), the amino acid or ester thereof may be any of a variety of natural or unnatural amino acids, including those discussed above in connection with Formula (1) in Reaction Scheme 1. The natural or unnatural amino acid or ester thereof may be selected, for example, from those which, when the moieties is attached to the cyclized compound, will affect certain interactions (e.g., electrostatic, hydrophobic, and/or mimetic interactions) between the composition and a target protein, affect solubility or other pharmacokinetic properties, affect the ability of the composition to penetrate cellular membranes, or function as a linking moiety for linking the cyclized six-membered heterocyclic 1,4-diaza-2-one (5) to a solid support or to another mimetic subunit, among other properties. Exemplary natural and unnatural amino acids or esters thereof include β-alanine and esters thereof (e.g., alkyl esters such as methyl esters, ethyl esters, etc.), aminoalkanoic acids (e.g., 3-aminobutanoic acid, 3-aminoisobutyric acids, and esters thereof), cycloalkylcarboxylic acids and esters thereof (e.g., cyclopentanecarboxylic acid, cyclohexanecarboxylic acid, and esters thereof), and the like.

Coupling

Other aspects of the present invention are directed to processes for the preparation of two, three, or more, six-membered heterocyclic 1,4-diaza-2-one moieties covalently bonded by a hydrazine bond. The processes generally involve treating a six-membered heterocyclic 1,4-diaza-2-one with a carboxylic acid in the presence of an activating agent.

The starting material for the coupling reaction is typically the six-membered heterocyclic 1,4-diaza-2-one (5), which may be synthesized in the manner described above. Various combinatorial synthetic methods are generally applicable to the preparation of the compositions of the present invention. These methods include, but are not limited to, solution phase synthesis methods, solid phase synthesis methods, and combinations thereof

For example, after the cyclization reaction described above in Stage 4 of Reaction Schemes 1, 2a and 2b, the six-membered heterocyclic 1,4-diaza-2-one (5) may be derivatized at the 1-aza position by connecting a linker moiety attached to a solid support (i.e., —Z₁). Alternatively, the carboxylic acid-substituted monoester (4a) and/or diacid (4b) may be cyclized as described above in Stage 4 of Reaction Schemes 1 and 2 in the presence of the linker moiety attached to the solid support, such that the six-membered heterocyclic 1,4-diaza-2-one (5) is attached to the solid support through the linker moiety, preferably at the 1-aza position (i.e., —Z₁).

In general, conventional (e.g., polymeric) solid supports and/or resins may be utilized in the cyclization and/or subsequent coupling reaction(s). Such solid supports generally possess pendant side chains bearing linking moieties which are generally reactive with the carboxylic acid-substituted monoester (4a) and/or diacid (4b) and/or the six-membered heterocyclic 1,4-diaza-2-one (5). Additionally, the linker moiety backbone is preferably inert to the carboxylic acid-substituted monoester (4a) and/or diacid (4b) or the six-membered 1,4-diaza-2-one (5), the activating agents employed, and/or the cyclizing and/or coupling reaction conditions under which the solid supports and/or resins will be used.

Various solid supports are known in the chemical synthesis art and include, for example, polystyrene (e.g., polystyrene utilizing 4-hydroxymethylphenoxybutyrate as a linker), polyoxyethylene-modified polystyrene, polyamide, and the like. Commercially available solid supports include, for example, TentaGel® (Rapp Polymere GmbH, Germany) or Controlled Pore Glass (Millipore, Billerica, Mass.). Often, the solid support includes or consists of a resin. Various commercially available solid supports including or consisting of resins are also known including, for example, those having a core of polystyrene or modified polystyrene (e.g., polyethylene glycol grafted polystyrene crosslinked with divinylbenzene; Argonaut Technologies, Inc. (San Carlos, Calif.)) utilizing aminomethyl polystyrene, benzhydrylamine (BHA) methylbenzhydrylamine (MBHA) (Matsueda et al., Peptides 2:45, 1981), phenoxybenzylalcohol (Wang resin) (Wang J. Am. Chem. Soc. 95, 1328, (1973)), 2-chlorotrytyl (Barlos et al., Tetrahedron Lett. 30, 3943, (1989), ibid 30:3947, 1989), PAL (Albericio et al., J. Org. Chem. 55, 3730, (1990)) resins, and the like. Other suitable solid supports are commercially available from such vendors as Polymer Laboratories Ltd. (UK), Advanced ChemTech (Louisville, Ky.), and Novabiochem (San Diego, Calif.).

Reaction Scheme 3 generally illustrates the process for preparing the compositions of the present invention, wherein R_{3A}, R_{3B}, R_{5A}, R_{5B}, R_{6A}, R_{6B}, R_{33A}, R_{33B}, R_{55A}, R_{55B}, R_{66A}, R_{66B}, R_{333A}, R_{333B}, R_{555A}, R_{555B}, R_{666A}, and R_{666B}, are as defined in connection with Formulae (50) and (60).

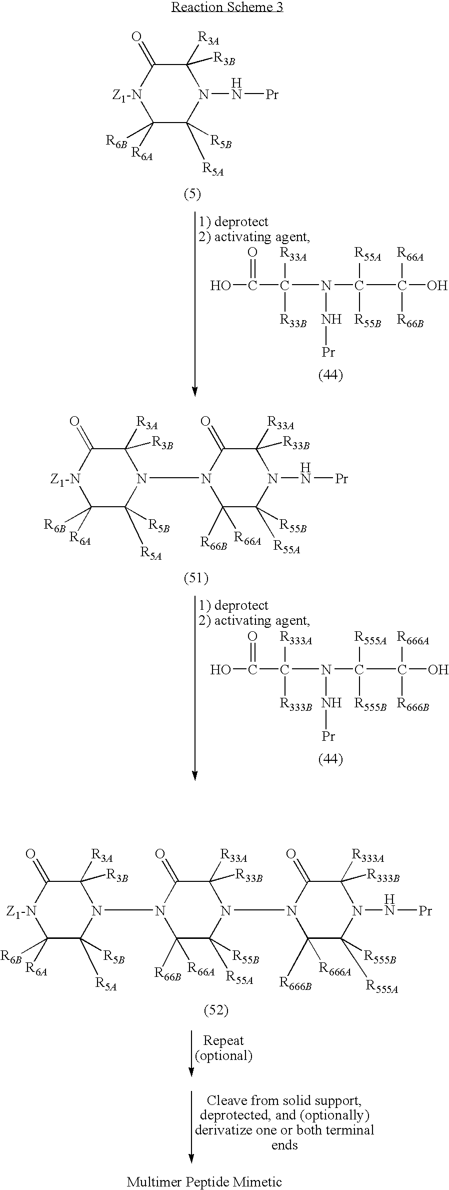

The process of Reaction Scheme 3 generally applies regardless of whether solid phase or solution phase synthesis methods are employed. For example, where solid phase methods are employed, Z₁ is a linker moiety attached to a solid support. Generally, solid support-linker coupled six-membered heterocyclic 1,4-diaza-2-one moieties are prepared by first removing the protecting group from the resin or other solid support to activate the functional group (e.g., an amino functional group), followed by a coupling reaction including the six-membered heterocyclic 1,4-diaza-2-one moiety. Suitable activating/coupling agents are described in detail above. Where solution phase methods are employed, Z₁ is typically another moiety selected from those defined in connection with Z₁—(W₁)ₙ—P₁-P₂—(W₂)ₘ—Z₂ above (e.g., hydrocarbyl, substituted hydrocarbyl, etc.).

The next step is the systematic or stepwise addition of additional six-membered heterocyclic 1,4-diaza-2-one moieties or other six-membered carbocyclic or heterocyclic moieties, at least one of which is preferably bonded to the previous moiety by a hydrazine bond. This generally involves the repeated deprotection/coupling cycles shown in Reaction Scheme 3. For example, the amino protecting group (Pr) is removed (i.e., deprotected) from the six-membered heterocyclic 1,4-diaza-2-one (5) by treatment with a hydrolyzing agent. Typically, the deprotection step is followed by neutralization with a base.

Any hydrolyzation agent may be utilized to deprotect the terminal amino moiety, provided it does not disturb any of the other substituents on the six-membered heterocyclic 1,4-diaza-2-one(s) (e.g., R_{3A}, R_{3B}, R_{5A}, R_{5B}, R_{6A}, R_{6B}, R_{33A}, R_{33B}, R_{55A}, R_{55B}, R_{66A}, R_{66B}, R_{333A}, R_{333B}, R_{555A}, R_{555B}, R_{666A}, and R_{666B}, and so on, and/or the hydrazine bonds), and/or detach the compound from the solid support and/or linker (i.e., —Z₁).

The hydrolyzation agent is typically an organic acid such as, for example, benzenesulfonic, ethanesulfonic, formic, naphthalenesulfonic, phosphonic, sulfamic, sulfanilic, toluenesulfonic, trichloroacetic, trifluoroacetic, trifluoromethanesulfonic acids, and the like, and mixtures thereof The base for the neutralization reaction is preferably an amine base, such as triethylamine (TEA), tributylamine, triethylenediamine, N,N′-dicyclohexylmethylamine, diisopropylamine, N,N′-diisopropylmethylamine, and the like, and mixtures thereof

The coupling reaction is performed by treating the six-membered heterocyclic 1,4-diaza-2-one (5) with the carboxylic acid (44) in the presence of an activating agent to cyclize the carboxylic acid (44) as described above so as to attach a second six-membered heterocyclic 1,4-diaza-2-one to the first by way of a hydrazine bond, thus forming compound (51). This deprotection/coupling cycle may then be repeated using another carboxylic acid (444) to form the three, six-membered heterocyclic 1,4-diaza-2-one moieties covalently bonded by hydrazine bonds (52). The carboxylic acid compounds may be prepared in the manner described above, or by some other method. The deprotection/coupling cycle may be repeated as many times as desired, not only using the carboxylic acid compounds corresponding to Formulae (4), (44), and (444), but using any compound known to cyclize and couple as described herein so as to form a six-membered carbocyclic or heterocyclic ring.

As noted above, the composition of the present invention include two, six-membered heterocyclic 1,4-diaza-2-one moieties covalently bonded by a hydrazine bond (i.e., P₁ and P₂), and generally include 1 to 10 six-membered carbocyclic or heterocyclic rings (i.e., W₁ and/or W₂). Thus, the deprotection/coupling cycle may be repeated anywhere from one to eleven times. Preferably, the deprotection/coupling cycle is performed at least once to form two, six-membered heterocyclic 1,4-diaza-2-one moieties covalently bonded by a hydrazine bond (i.e., P₁ and P₂); more preferably, the deprotection/coupling cycle is performed at least twice.

Upon the formation of a composition of desired length, it may be cleaved from the resin, deprotected, and optionally derivatized at one or both terminal ends, thus forming a peptido- and/or proteomimetic compound.

It will be understood that the process illustrated in Reaction Scheme 3 may be carried out in the same manner where a hydrazine amide intermediate (4aa) as described in connection with Reaction Scheme 2b is employed instead of the diacid-substituted carboxylic acid (44) and (444) in the cyclization and coupling reactions. That is, monoesters generally corresponding to formula (4a) and diacids generally corresponding to formula (4b) may be used interchangeably in the formation of multimer peptide mimetics according to the cyclization and coupling reaction illustrated in Reaction Scheme 3 (see, e.g., monoester (44a) and diacid (44b), above). In the embodiments in which the cyclized monomer subunits are bonded together by a hydrazine bond, however, diacid (4b) precursors are preferably employed following the formation of the initial cyclized subunit.

Reaction Scheme 4 illustrates the derivatization of three, six-membered heterocyclic 1,4-diaza-2-one moieties covalently bonded by hydrazine bonds, wherein R_{3A}, R_{3B}, R_{5A}, R_{5B}, R_{6A}, R_{6B}, R_{33A}, R_{33B}, R_{55A}, R_{55B}, R_{66A}, R_{66B}, R_{333A}, R_{333B}, R_{555A}, R_{555B}, R_{666A}, and R_{666B} are as defined in connection with Formulae (50) and (60).

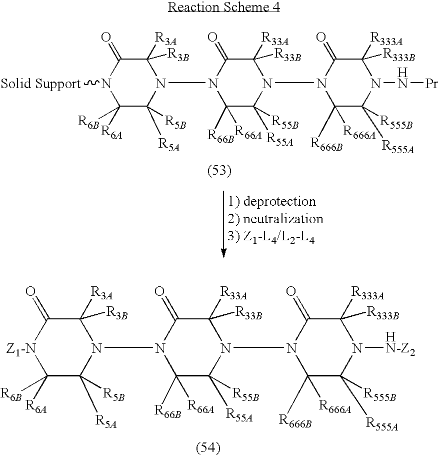

Following the formation of a composition of desired length, the hydrazine-bonded, six-membered heterocyclic 1,4-diaza-2-one moieties can be cleaved from the linker (represented by the wavy line connected to the nitrogen atom) attached to the solid support, or other moiety at Z₁, deprotected at the opposite terminal end (i.e., the Pr moiety is removed) by treatment with a hydrolyzing agent, and optionally derivatized at either one or both terminal ends according to conventional methods.

Depending on the particular hydrolyzing agent employed, the amino protecting group (Pr) may be removed and the resulting amino moiety selectively derivatized without removing the composition from the solid support (e.g., using a weaker acid), or the composition may be simultaneously or substantially simultaneously cleaved from the solid support at one terminal end and deprotected at the other terminal end (e.g., using a stronger acid such as trifluoromethanesulfonic acid) and selectively derivatized at both ends. The deprotection reaction is typically neutralized with a base following deprotection, as described above. The hydrolyzing agent is preferably an organic acid, and suitable organic acids hydrolyzing agents are described above.

Following the removal of the protecting group (Pr) and/or the cleavage of the composition from the resin, the composition may be derivatized by treatment with Z₁-L₄ and/or Z₂-L₄ to attach —Z₁ and/or —Z₂ moieties at the terminal ends, wherein Z₁ and Z₂ are as defined above in connection with Z₁—(W₁)ₙ—P₁-P₂—(W₂)ₘ—Z₂ above. Generally, Z₁ and Z₂ may be the same or different and, as noted above, are typically included on the composition to affect certain interactions (e.g., electrostatic, hydrophobic, and/or mimetic interactions) between the composition and a target protein, affect solubility or other pharmacokinetic properties, and/or affect the ability of the composition to penetrate cellular membranes. For instance, Z₁-L₄ and/or Z₂-L₄ may be protected carboxylates of varying lengths. The leaving group (L₄) of the reagents used to attach the Z₁ and/or Z₂ moieties to the composition can be, for example, chloro, bromo, iodo, benzyl, methanesulfonyloxy, trifluoromethanesulfonyloxy, 2,2,2-trifluoroethanesulfonyloxy, benzenesulfonyloxy, p-bromobenzenesulfonyloxy, p-nitrobenzenesulfonyloxy or p-toluenesulfonyloxy, and the like. Alternatively, depending on the mode of synthesis, the Z₁ and/or Z₂ substituents may be selected to remain as opposed to replacing them with alternative substituents.

For convenience purposes, the three, six-membered heterocyclic 1,4-diaza-2-one moieties covalently bonded by hydrazine bonds are shown connected to the solid support by a linker (the linker being represented by the wavy line). Alternatively, however, the derivatization process shown in Reaction Scheme 4 may apply where conventional solution phase synthesis methods are employed.

Pharmaceutical Compositions and Methods for Treatment and Prophylaxis

In certain aspects of the present invention, compositions according to the present invention may be used as active agents in pharmaceutical compositions as agonists or inhibitors of alpha-helical proteins in their interactions with proteins (such as receptors, enzymes, other proteins) or other binding sites, said compositions comprising an effective amount of one or more of the compositions disclosed above, formulated as a pharmaceutical dosage form, optionally in combination with a pharmaceutically acceptable carrier, additive or excipient. Pharmaceutical compositions according to the present invention may be used in the treatment of cancer (as, for example, a suppressor of Mdm2/p53 tumor, to inhibit BcL protein family/Bak protein family or AP-1 transcription factor/DNA complex), proliferative diseases including, for example, psoriasis, genital warts and hyperproliferative keratinocyte diseases including hyperkeratosis, ichthyosis, keratoderma or lichen planus, neuropeptide Y receptor interactions, including the resulting hypertension and neuronal/neurological effects (to facilitate neuromodulation through, for example, inhibition of calmodulin binding on calmodulin dependent phosphodiesterase including PDE1A, PDE1B and PDE1C, among others), neurodegenerative diseases including Alzheimer's disease and Parkinson's disease, Herpes simplex virus infections (e.g., HSV, through inhibition of the HSV VP16/human TAF1131 HSV infection complex), HIV infections (e.g., through inhibition of HIVp7 nuclear capsid protein/RNA interaction or alternatively, through inhibition of the REV protein RNA complex), asthma, hypertension, cancer and autoimmune diseases (e.g., through immunomodulation, for example, by inhibition or modulation of interleukin/receptor interaction), numerous viral infections other than HIV or HSV through inhibition of ribonucleotide reductase dimerization, or to modulate nuclear receptor/coactivator protein complex interaction (e.g., estrogen receptor for anticancer therapy) and to disrupt G protein coupled receptor (GPCR) function (e.g., through displacement of one of the helixes and disruption of the helix packing interactions or alternatively, by blocking the interaction of the ligand with GPCR, e.g., where the ligand contains a key helix binding domain (e.g., GCSF, calcitonin, interleukins, parathyroid hormones, among others).

Generally speaking, the binding sites refer to one or more sites at which an alpha-helical protein binds and elicits some response or action at that site, which action may be direct or indirect. Compositions according to the present invention may also bind at the binding site of the alpha-helical binding site in a manner which is agonistic or antagonistic. The binding site may be, for example, another protein, a receptor (such as a cell surface receptor or a G-protein coupled receptor), signaling proteins, proteins involved in apoptotic pathways (e.g., neuronal apoptosis), active sites and regulatory domains of enzymes, growth factors, DNA, RNA (including polynucleotides and oligonucleotides), viral fusion proteins, and viral coat proteins, among a number of others.

In other aspects of the present invention, certain compositions according to the present invention may be used as agonists or antagonists in binding assays, as analytical agents, as agents to be used to isolate or purify proteins, and as intermediates in the synthesis of further peptido- and/or proteomimetic agents, among other uses.

The present invention also relates to methods for the treatment and prophylaxis of diseases, pathological disorders, and/or medical conditions which are modulated through interactions between alpha-helical proteins and other proteins or binding sites; that is, the binding or lack or absence of binding of an alpha-helical protein to a binding site produced or will produce, either directly or indirectly, a disease, pathological disorder, and/or medical condition with is sub-optimal and in many cases, debilitating and even life threatening. Thus, in one method aspect of the present invention, pharmaceutical compositions comprising alpha-helical protein agonists or antagonists may be used to treat any diseases, pathological disorders, and/or medical condition in which alpha-helical proteins modulate their activity through a receptor or other binding site. Thus, for example, the present invention may relate to methods for the inhibition of protein binding to binding sites within the patient in order to affect a biological/pharmacological result. Compositions according to the present invention may be used as proteomimetics to inhibit the interaction between a native alpha-helical protein (i.e., a natural alpha-helical protein normally found in a patient) and its binding site. Preferred compositions according to the present invention may be used to disrupt or compete with the binding of a number of proteins including, for example, calmodulin (CaM) with binding sites on smooth muscle light chain kinase (smMLCK) or phosphodiesterase (PDE1A, PDE1B, PDE1C) with resulting neuromuscular and neuronal (among other) effects in the treating of disease states or conditions, gp41 (HIV) and other viruses such as HSV or HBV, for the viral invasive binding cites in CD4 and/or other hematopoietic cells, genital/mucosal cells, among others (HSV) and hepatocytes (HBV), among numerous others and pro-apoptotic Bak- and/or Bad-proteins, for their binding interaction with Bcl-x_{L} protein in a preferred treatment for cancer.

Thus, the present invention is directed to the treatment and prophylaxis of diseases, pathological disorders, and/or medical conditions which are modulated through interactions between alpha-helical proteins and other proteins or binding sites of the alpha-helical proteins selected from the group consisting of viral infections (including Hepatitis B virus (HBV) infections, human immunodeficiency virus (HIV) infections or conditions associated with such infections (AIDS), Herpes Simplex virus infections (HSV) infections, tumors and/or cancer, proliferative diseases including psoriasis, genital warts and hyperproliferative keratinocyte diseases including hyperkeratosis, ichthyosis, keratoderma, lichen planus, hypertension, neuronal disorders by promoting neuromodulation including, for example, attention deficit disorder, memory loss, language and learning disorders, asthma, autoimmune diseases including lupus (lupus erythematosus), multiple sclerosis, arthritis, including rheumatoid arthritis, rheumatic diseases, fibromyalgia, Sjogren's disease and Grave's disease and neurodegenerative diseases including Alzheimer's disease and Parkinson's disease, said method comprising administering to a patient in need thereof an effective amount of a pharmaceutical composition comprising any one or more of the compositions previously described above.

Aspects of the present invention include compositions which have been described in detail hereinabove or to pharmaceutical compositions which comprise an effective amount of one or more compositions according to the present invention, optionally in combination with a pharmaceutically acceptable carrier, additive or excipient (described in further detail below).

Another aspect of the present invention is directed to compositions according to the present invention which may be used to mimic alpha-helical proteins in an agonistic or antagonistic manner. In this aspect of the present invention, one or more of the compositions according to the present invention may be used to mimic or inhibit the binding of an alpha-helical protein for its binding site, whether that binding site is another protein, a receptor (such as a cell surface receptor or a G-protein coupled receptor), signaling proteins, proteins involved in apoptotic pathways (especially neuronal apoptosis), active sites and regulatory domains of enzymes, growth factors, DNA, RNA (including oligonucleotides), viral fusion proteins and viral coat proteins, among numerous others. In certain aspects of the present invention, one or more compound according to the present invention may be used to inhibit the binding of calmodulin to a calmodulin dependent phosphodiesterase enzyme (PDE1A, PDE1B or PDE1C).

Dosage and Amount and Time Course of Treatment

The dose or amount of pharmaceutical compositions including the compositions of the present invention administered to the mammal should be an effective amount for the intended purpose, i.e., treatment or prophylaxis of one or more of the diseases, pathological disorders, and medical conditions noted above. Generally speaking, the effective amount of the composition administered to the mammal can vary according to a variety of factors such as, for example, the age, weight, sex, diet, route of administration, and the medical condition of the mammal Specifically preferred doses are discussed more fully below. It will be understood, however, that the total daily usage of the compositions described herein will be decided by the attending physician or veterinarian within the scope of sound medical judgment.

The specific therapeutically effective dose level for any particular mammal will depend upon a variety of factors including the disorder being treated and the severity of the disorder; activity of the specific compound(s) employed; the age, body weight, general health, sex and diet of the patient; the time of administration; the route of administration; the rate of excretion of the specific compound(s) employed; the duration of the treatment; drugs used in combination or coincidental with the specific compound(s) employed and like factors well known in the medical and/or veterinary arts. For example, it is well within the skill of the art to start doses of the compound(s) at levels lower than those required to achieve the desired therapeutic effect and to gradually increase the dosage until the desired effect is achieved. If desired, the effective daily doses may be divided into multiple doses for purposes of administration. Consequently, single dose compositions may contain such amounts or submultiples to make up the daily dose.

Administration of the pharmaceutical composition can occur as a single event or over a time course of treatment. For example, one or more of the compositions of the present invention can be administered hourly (e.g., every hour, every two hours, every three hours, every four hours, every five hours, every six hours, and so on), daily, weekly, bi-weekly, or monthly. For treatment of acute conditions, the time course of treatment may be at least several hours or days. Certain conditions could extend treatment from several days to several weeks. For example, treatment could extend over one week, two weeks, or three weeks. For more chronic conditions, treatment could extend from several weeks to several months, a year or more, or the lifetime of the mammal in need of such treatment. Alternatively, the compositions can be administered hourly, daily, weekly, bi-weekly, or monthly, for a period of several weeks, months, years, or over the lifetime of the mammal as a prophylactic measure.

One or more of the compositions of the present invention may be utilized in a pharmaceutically acceptable carrier, additive or excipient at a suitable dose ranging from about 0.05 to about 100 mg/kg of body weight per day, preferably within the range of about 0.1 to 50 mg/kg/day, most preferably in the range of 1 to 20 mg/kg/day. As noted above, the desired dose may conveniently be presented in a single dose or as divided doses administered at appropriate intervals, for example as two, three, four or more sub-doses per day.

Ideally, the active ingredient should be administered to achieve effective peak plasma concentrations of the active compound within the range of from about 0.05 uM to about 5 uM. This may be achieved, for example, by the intravenous injection of about a 0.05 to 10% solution of the active ingredient, optionally in saline, or orally administered as a bolus containing about 1 mg to about 5 g, preferably about 5 mg to about 500 mg of the active ingredient, depending upon the active compound and its intended target. Desirable blood levels may be maintained by a continuous infusion to preferably provide about 0.01 mg/kg/hour to about 2.0 mg/kg/hour or by intermittent infusions containing about 0.05 mg/kg to about 15 mg/kg of the active ingredient. Oral dosages, where applicable, will depend on the bioavailability of the compositions from the GI tract, as well as the pharmacokinetics of the compositions to be administered. While it is possible that, for use in therapy, one or more compositions of the invention may be administered as the raw chemical, it is preferable to present the active ingredient as a pharmaceutical formulation, presented in combination with a pharmaceutically acceptable carrier, excipient or additive.

Routes of Administration, Formulations/Pharmaceutical Compositions

As noted above, the above-described compositions of the present invention may be dispersed in a pharmaceutically acceptable carrier prior to administration to the mammal The carrier, also known in the art as an excipient, vehicle, auxiliary, adjuvant, or diluent, is typically a substance which is pharmaceutically inert, confers a suitable consistency or form to the composition, and does not diminish the efficacy of the compound. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier will also, in part, be a function of the route of administration. In general, the compositions of the present invention can be formulated for any route of administration so long as the blood circulation system is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable carriers for use in combination with the compositions of the present invention are well known to those of ordinary skill in the art and are selected based upon a number of factors: the particular compound used, and its concentration, stability and intended bioavailability; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2 to 30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)-lactamide, N,N-dimethylacetamide_amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di-, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di-, or tri-glycerides, fatty acid esters such as isopropyl myristrate, fatty acid derived PEG esters such as PEG-hydroxyoleate and PEG-hydroxystearate, N-methyl pyrrolidinone, pluronic 60, polyoxyethylene sorbitol oleic polyesters such as poly(ethoxylated)₃₀₋₆₀ sorbitol poly(oleate)_{2-4,} poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄ to C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2 to 30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3 to 30 carbon atoms (e.g., acetone, methyl ethyl ketone, methyl isobutyl ketone); aliphatic, cycloaliphatic or aromatic hydrocarbons having 4 to 30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1 to 30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), and A. J. Spiegel et al., Use of Nonaqueous Solvents in Parenteral Products, Journal of Pharmaceutical Sciences, Vol. 52, No. 10, pp. 917-927 (1963).

Formulations containing the compositions of the present invention may take the form of solid, semi-solid, lyophilized powder, or liquid dosage forms such as, for instance, aerosols, capsules, creams, emulsions, foams, gels/jellies, lotions, ointments, pastes, powders, soaps, solutions, sprays, suppositories, suspensions, sustained-release formulations, tablets, tinctures, transdermal patches, and the like, preferably in unit dosage forms suitable for simple administration of precise dosages.

Salts and Prodrugs

As noted above, the pharmaceutical compositions may include compositions of the present invention in their salt form. Typically, the salt will be a pharmaceutically acceptable salt; that is, a salt prepared from pharmaceutically acceptable non-toxic acids, including inorganic acids and organic acids. Suitable non-toxic acids include inorganic and organic acids of basic residues such as amines, for example, acetic, benzenesulfonic, benzoic, amphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, aluminum hydroxide, lithium hydroxide, magnesium hydroxide, zinc hydroxide, ammonia, trimethylammonia, triethylammonia, ethylenediamine, lysine, arginine, ornithine, choline, N,N″-dibenzylethylenediamine, chloroprocaine, diethanolamine, procaine, n-benzylphenethylamine, diethylamine, piperazine, tris(hydroxymethyl)-aminomethane, tetramethylammonium hydroxide, and the like. Pharmaceutically acceptable salts of the compositions described herein can be prepared by reacting the free acid or base forms of these compositions with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, each of which is hereby incorporated by reference herein.

Since prodrugs are known to enhance numerous desirable pharmaceuticals (e.g., solubility, bioavailability, manufacturing), the compound(s) may be delivered in prodrug form. Thus, the present invention is intended to cover prodrugs of the compositions described above, methods of delivering the same and compositions containing them. Prodrugs generally include any covalently bonded carriers which release an active parent drug in vivo when such prodrug is administered to a mammalian subject. Prodrugs are generally prepared by modifying functional groups present in the compound in such a way that the modifications are cleaved, either in routine manipulation or in vivo, to the parent compound. Prodrugs include compositions of the present invention wherein a hydroxyl or amino group is bonded to any group that, when the prodrug is administered to a mammalian subject, cleaves to form a free hydroxyl or free amino group, respectively. Examples of prodrugs include, but are not limited to, acetate, formate, and benzoate derivatives of alcohol and amine functional groups in the compositions and conjugates of the present invention. Prodrugs of the compound are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compositions of the invention. Prodrugs may refer to compounds that are rapidly transformed in vivo to yield the compound(s) of the present invention, for example by hydrolysis in blood. A thorough discussion of prodrugs is provided in the following: Design of Prodrugs, H. Bundgaard, ea., Elsevier, 1985; Methods in Enzymology, K. Widder et al, Ed., Academic Press, 42, p.309-396, 25 1985; A Textbook of Drug Design and Development, Krogsgaard-Larsen and H. Bundgaard, ea., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, each of which is hereby incorporated by reference herein.

Additional Pharmaceutical Components

The above-described pharmaceutical compositions including the compositions of the present invention may additionally include one or more pharmaceutically active components. Suitable pharmaceutically active agents that may be included in the compositions of the present invention include, for instance, anesthetics, antihypertensives, antianxiety agents, anticlotting agents, anticonvulsants, blood glucose-lowering agents, decongestants, antihistamines, antitussives, antineoplastics, beta blockers, anti-inflammatory agents, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, anti-depressants, and antiviral agents.

The individual components of such combinations may be administered either sequentially or simultaneously in separate or combined pharmaceutical formulations.

### Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

The terms “acetal” and “ketal,” as used herein alone or as part of another group, denote the moieties represented by the following formulae, respectively:

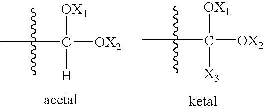

wherein X₁ and X₂ are independently hydrocarbyl, substituted hydrocarbyl, heterocyclo, or heteroaryl, and X₃ is hydrocarbyl or substituted hydrocarbyl, as defined in connection with such terms, and the wavy lines represent the attachment point of the acetal or ketal moiety to another moiety or compound.

The term “acyl,” as used herein alone or as part of another group, denotes the moiety formed by removal of the hydroxy group from the group —COOH of an organic carboxylic acid, e.g., X₄C(O)—, wherein X₄ is X¹, X¹O—, X¹X²N—, or X¹S—, X¹ is hydrocarbyl, heterosubstituted hydrocarbyl, or heterocyclo, and R² is hydrogen, hydrocarbyl or substituted hydrocarbyl. Exemplary acyl moieties include acetyl, propionyl, benzoyl, pyridinylcarbonyl, and the like.

The term “acyloxy,” as used herein alone or as part of another group, denotes an acyl group as described above bonded through an oxygen linkage (—O—), e.g., XC(O))— wherein X is as defined in connection with the term “acyl.”

The term “alkanol,” as used herein alone or as part of another group, denotes an alkyl radical having 1 to 10 carbon atoms, which is substituted by one, two or three, or more, hydroxyl group(s). Examples of alkanols include methanol, ethanol, n-propan-2-ol, n-propan-3-ol, isopropanol, i-butanol, and the like.

The term “alkoxy,” as used herein alone or as part of another group, denotes an —OX₅ radical, wherein X₅ is as defined in connection with the term “alkyl.” Exemplary alkoxy moieties include methoxy, ethoxy, propoxy, or 2-propoxy, n-, iso-, or tert-butoxy, and the like.

The term “alkenoxy,” as used herein alone or as part of another group, denotes an —OX₆ radical, wherein X₆ is as defined in connection with the term “alkenyl.” Exemplary alkenoxy moieties include ethenoxy, propenoxy, butenoxy, hexenoxy, and the like.

The term “alkynoxy,” as used herein alone or as part of another group, denotes an —OX₇ radical, wherein X₇ is as defined in connection with the term “alkynyl.” Exemplary alkynoxy moieties include ethynoxy, propynoxy, butynoxy, hexynoxy, and the like.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkylene,” as used herein alone or as part of another group, denotes a linear saturated divalent hydrocarbon radical of one to eight carbon atoms or a branched saturated divalent hydrocarbon radical of three to six carbon atoms unless otherwise stated. Exemplary alkylene moieties include methylene, ethylene, propylene, 1-methylpropylene, 2-methylpropylene, butylene, pentylene, and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “amine” or “amino,” as used herein alone or as part of another group, represents a group of formula —N(X₈)(X₉), wherein X₈ and X₉ are independently hydrogen, hydrocarbyl, substituted hydrocarbyl, heteroaryl, or heterocyclo, or X₈ and X₉ taken together form a substituted or unsubstituted alicyclic, aryl, or heterocyclic moiety, each as defined in connection with such term, typically having from 3 to 8 atoms in the ring. “Substituted amine,” for example, refers to a group of formula —N(X₈)(X₉), wherein at least one of X₈ and X₉ are other than hydrogen. “Unubstituted amine,” for example, refers to a group of formula —N(X₈)(X₉), wherein X₈ and X₉ are both hydrogen.

The terms “amido” or “amide,” as used herein alone or as part of another group, represents a group of formula —CON(X₈)(X₉), wherein X₈ and X₉ are as defined in connection with the terms “amine” or “amino ” “Substituted amide,” for example, refers to a group of formula —CON(X₈)(X₉), wherein at least one of X₈ and X₉ are other than hydrogen. “Unsubstituted amido,” for example, refers to a group of formula —CON(X₈)(X₉), wherein wherein X₈ and X₉ are both hydrogen.

The terms “amino protecting group,” “protected amino,” or “Pr” as used herein denote moieties that block reaction at the protected amino group while being easily removed under conditions that are sufficiently mild so as not to disturb other substituents of the various compounds. Exemplary amino protecting groups include benzyl, benzoyl, carbobenzyloxy (Cbz), t-butoxycarbonyl (Boc or t-Boc), allyloxycarbonyl, fluorenylmethoxycarbonyl (Fmoc), and the like. A variety of protecting groups for the amino group and the synthesis thereof may be found in “Protective Groups in Organic Synthesis” by T. W. Greene and P. G. M. Wuts, John Wiley & Sons, 1999.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “arylene”, as used herein alone or part of another group refers to a divalent aryl radical of one to twelve carbon atoms. Non-limiting examples of “arylene” include phenylene, pyridinylene, pyrimidinylene and thiophenylene.

The terms “aralkyl,” “arylalkyl,” or “alkylene aryl,” as used herein alone or as part of another group, denotes an -(alkylene)-X₁₀ radical, wherein X₁₀ is as defined in connection with the term “aryl.” Non-limiting examples of “aralkyl” or “alkylene aryl” moieties include benzyl, —(CH₂)ₙ-phenyl where n is 2 to 6, or —CH-(phenyl)₂.

The terms “alkaryl” or “alkylaryl,” as used herein alone or as part of another group, denotes an -(arylene)-X₁₁ radical, wherein X₁₁ is as defined in connection with the term “alkyl.”

The term “carbocyclic,” as used herein alone or as part of another group, denotes a ring wherein the atoms forming the ring backbone are selected from only carbon atoms. The carbocyclic rings may be optionally substituted, fully saturated or unsaturated, monocyclic or bicyclic, aromatic or nonaromatic, and generally include 3 to 20 carbon atoms.

The term “cyano,” as used herein alone or as part of another group, denotes a group of formula —CN.

The term “cycloalkyl,” as used herein alone or as part of another group, denotes a cyclic saturated monovalent bridged or non-bridged hydrocarbon radical of three to ten carbon atoms. Exemplary cycloalkyl moieties include cyclopropyl, cyclobutyl, cyclopentyl, cyclohexyl, or adamantyl. Additionally, one or two ring carbon atoms may optionally be replaced with a —CO— group.

The term “ester,” as used herein alone or as part of another group, denotes a group of formula —COOX₁₂ wherein X₁₂ is alkyl or aryl, each as defined in connection with such term.

The term “ether,” as used herein alone or as part of another group, includes compounds or moieties which contain an oxygen atom bonded to two carbon atoms. For example, ether includes “alkoxyalkyl” which refers to an alkyl, alkenyl, or alkynyl group substituted with an alkoxy group.

The terms “halide,” “halogen” or “halo” as used herein alone or as part of another group refer to chlorine, bromine, fluorine, and iodine.

The term “heteroatom” shall mean atoms other than carbon and hydrogen.

The terms “heteroaralkyl” and “alkylene heteroaryl,” as used herein alone or as part of another group, denotes an -(alkylene)-X₁₃ radical, wherein X₁₃ is as defined in connection with the term “heteroaryl.” Non-limiting examples of “heteroaralkyl” or “alkylene heteroaryl” moieties include —(CH₂)ₙ-indolyl where n is 1 to 6.

The term “heteroaromatic” or “heteroaryl” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “hydroxy,” as used herein alone or as part of another group, denotes a group of formula —OH.

The term “hydroxy protecting group,” as used herein alone or as part of another group, denote a group capable of protecting a free hydroxy group (“protected hydroxy”) which, subsequent to the reaction for which protection is employed, may be removed without disturbing the remainder of the molecule. Exemplary hydroxy protecting groups include ethers (e.g., allyl, triphenylmethyl (trityl or Tr), benzyl, p-methoxybenzyl (PMB), p-methoxyphenyl (PMP)), acetals (e.g., methoxymethyl (MOM), β-methoxyethoxymethyl (MEM), tetrahydropyranyl (THP), ethoxy ethyl (EE), methylthiomethyl (MTM), 2-methoxy-2-propyl (MOP), 2-trimethylsilylethoxymethyl (SEM)), esters (e.g., benzoate (Bz), allyl carbonate, 2,2,2-trichloroethyl carbonate (Troc), 2-trimethylsilylethyl carbonate), silyl ethers (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like. A variety of protecting groups for the hydroxy group and the synthesis thereof may be found in “Protective Groups in Organic Synthesis” by T. W. Greene and P. G. M. Wuts, John Wiley & Sons, 1999.

The term “keto,” as used herein alone or as part of another group, denotes a double bonded oxygen moiety (i.e., ═O).

The term “nitro,” as used herein alone or as part of another group, denotes a group of formula —NO₂.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, ethers, and thioethers.

The term “thioester,” as used herein alone or as part of another group, denotes a group of formula —C(O)—S—X₁₄, wherein X₁₄ is alkyl or aryl as defined in connection with such term.

The term “thioether,” as used herein alone or as part of another group, denotes compounds and moieties that contain a sulfur atom bonded to two different carbon or hetero atoms (i.e., -S-), and also includes compounds and moieties containing two sulfur atoms bonded to each other, each of which is also bonded to a carbon or hetero atom (i.e., dithioethers (—S—S—)). Examples of thioethers include, but are not limited to, alkylthioalkyls, alkylthioalkenyls, and alkylthioalkynyls. The term “alkylthioalkyls” includes compounds with an alkyl, alkenyl, or alkynyl group bonded to a sulfur atom that is bonded to an alkyl group. Similarly, the term “alkylthioalkenyls” and alkylthioalkynyls” refer to compounds or moieties where an alkyl, alkenyl, or alkynyl group is bonded to a sulfur atom that is covalently bonded to an alkynyl group.

The term “thiol,” as used herein alone or as part of another group, denotes a group of formula —SH.

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Alpha-Amino Ester Formation

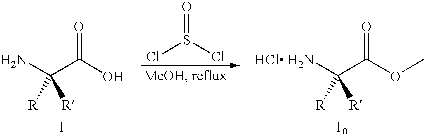

All amino esters (1a, b, c, d, e, f, g, h,) were prepared using the following procedure, except for phenylalanine methyl ester, 1a, which was obtained commercially. The free amine or the N-Boc protected amino acid 1 (1.59 mmol) was dissolved in MeOH (5 mL) and cooled to 0° C. Thionyl chloride (3.17 mmol, 2 equiv.) was added slowly and stirred at room temperature for 6 hours; then the mixture was refluxed for 12 hours. The solvent was removed under reduced pressure giving compound 1₀ as a white powder. NMR data for certain of the compounds is as follows:

Methyl 2-amino-2-methylpropanoate hydrochloride (1f). 93% yield. ¹H NMR (400 MHz, (CD₃)₂SO) δ 1.48 (s, 6H), 3.75 (s, 3H), 8.76 (br s 2H). ESI-MS 118 [M+H]⁻.

(R)-methyl 2-amino-3-(tert-butyldisulfanyl)propanoate hydrochloride (1 g). 96% yield.¹H NMR (400 MHz, CD₃OD) δ 1.37 (s, 9H), 3.18 and 3.28 (2dd, 2H), 3.86 (s, 3H), 4.37-4.40 (m, 1H). ¹³C NMR (100 MHz, CD₃OD) δ 23.02, 28.91, 39.32, 51.86, 52.74, 171.48. ESI-MS 224.1 [M+H]⁺.

(S)-methyl 2-amino-3-(naphthalene-2-yl)propanoate hydrochloride (1 h). 97% yield. ¹H NMR (400 MHz, (CD₃)₂SO) δ 3.28 (dd, J=12.4 and 4.4 Hz, 1H), 3.37 (dd, J=14.4 and 6.0 Hz, 1H), 3.69 (s, 3H), 4.41 (t, J=6.6 Hz, 1H), 7.49-7.54 (m, 2H), 7.38 (d, J=8.4 Hz, 1H), 7.76 (s, 1H), 7.86-7.92 (m, 3H), 8.58 (br, 2H). ¹³C NMR (100 MHz, (CD₃)₂SO) δ 35.93, 52.60, 54.81, 125.88, 126.16, 127.26, 127.43, 127.49, 128.11, 128.15, 132.04, 132.13, 132.87, 169.32. ESI-MS 230.1 [M+H]⁺.

### Example 2A

### Diester Synthesis

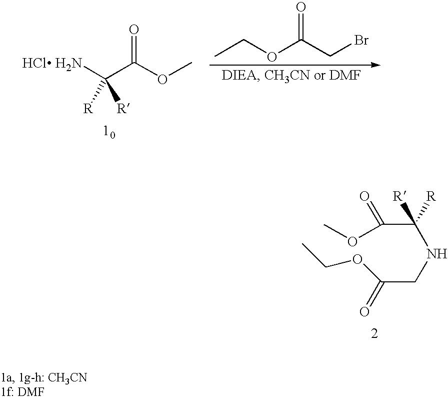

The HCl salt of amino esters 1a, 1g-h (4.63 mmol) were dissolved in dry CH₃CN (20 mL) and if was dissolved in DMF. Then DIEA (9.26 mmol, 2 equiv.) and ethyl bromoacetate (6.94 mmol, 1.5 equiv.) were added, while the reaction mixture was kept under argon; then it was stirred at room temperature for 24 hours. The reaction mixture was quenched with 5% citric acid (5 mL) and was extracted with ethyl acetate (3×15 mL). The combined organic layers were washed with brine (2×15 mL), dried over Na₂SO₄, and evaporated in vacuo to yield pure compounds 2a, 2g-h as colorless oils. Column chromatography (3:7 ethyl acetate: hexanes) was performed for crude 2f giving the desired product as a colorless oil.

(S)-methyl 2-(2-ethoxy-2-oxoethylamino)-3-phenylpropanoate (2a). R_{f}=0.32 (3:7 ethyl acetate:hexanes); 88% yield. ¹H NMR (400 MHz, CDCl₃) δ 1.23 (t, J=6.8 Hz, 3H), 2.11 (br s, 1H), 2.97 (dd, J=13.6 and 7.2 Hz, 1H), 3.04 (dd, J=13.4 and 6 Hz, 1H), 3.33 and 3.41 (2d, J=17.2 Hz, 2H), 3.66 (s, 3H), 4.14 (q, J=7.2 Hz, 2H), 7.18-7.30 (m, 5H). ²³C NMR (100 MHz, CDCl₃) δ 14.38, 39.74, 49.39, 52.04, 61.06, 62.37, 127.08, 128.73, 129.40, 137.12, 171.80, 174.29. ESI-MS 266.1 [M+H]⁺.

Methyl 2-(2-ethoxy-2-oxoethylamino)-2-methylpropanoate (20. Rf: 0.27 (3:7 ethyl acetate:hexanes); 60% yield. ¹H NMR (400 MHz, CDCl₃) δ 1.25 (t, J=7.0 Hz, 3H), 1.34 (s, 6H), 3.35 (s, 2H), 3.68 (s, 3H), 4.17 (q, J=7.2 Hz, 2H). ¹³C NMR (100 MHz, CDCl₃) δ 14.36, 25.34, 46.25, 52.34, 58.93, 61.25, 171.83, 176.62. ESI-MS 204.1 [M+H]⁺.

(R)-methyl 3-(tert-butyldisulfanyl)-2-(2-ethoxy-2-oxoethylamino)propanoate (2g). 80% yield. ¹H NMR (400 MHz, CDCl₃) δ 1.26 (t, J=7.2 Hz, 3H), 1.32 (s, 9H), 3.48 (d, J=7.2 Hz, 2H), 3.68 (t, J=6.2 Hz, 1H), 3.75 (s, 3H), 4.18 (q, J=7.2 Hz, 2H). ¹³ C NMR (100 MHz, CDCl₃) δ 14.40, 30.08, 43.41, 48.35, 49.28, 52.48, 60.29, 61.27, 171.53, 172.97. ESI-MS 310.1 [M+H]⁺.

### Example 2B

### Diester Synthesis

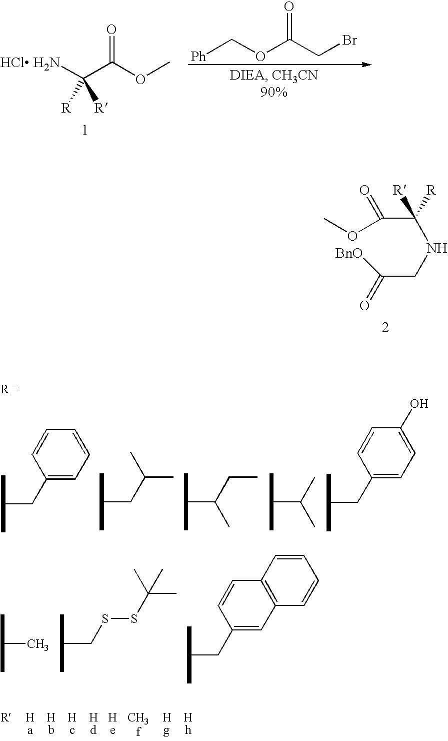

The HCl salt of amino ester 1a (2.32 mmol) was dissolved in dry CH₃CN (10 mL) under argon. Then, DIEA (4.63 mmol, 2 equiv.) and benzyl bromoacetate (3.47 mmol, 1.5 equiv.) were added, while the reaction mixture was kept under argon; then it was stirred at room temperature for 24 hours. The reaction mixture was quenched with 5% citric acid (5 mL) and was extracted with ethyl acetate (3×15 mL). The combined organic layers were washed with water and brine (2×15 mL), dried over Na₂SO₄, and evaporated in vacuo to yield compound 2a as a crude yellow oil. Column chromatography (3:7 ethyl acetate: hexanes) was performed to obtain the desired diester as a colorless oil. This procedure was repeated for sames 1b-1h.

### Example 3A

### Hydrazine Formation

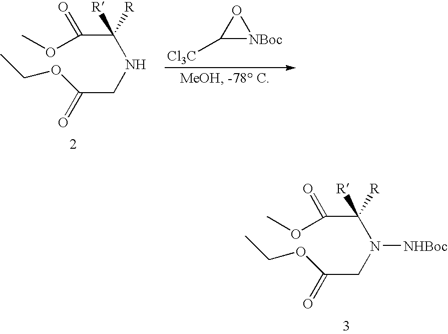

A solution of diester 2 (1.88 mmol) in dry methanol (7 mL) was cooled to −78° C. and kept under argon atmosphere. Tert-butyl 3-(trichloromethyl)-1,2-oxaziridine-2-carboxylate (5.64 mmol, 3 equiv.) was added slowly and the reaction was stirred at −78° C. for 4 hours. Progress of the reaction was monitored by TLC. The solvent was evaporated under reduced pressure and the crude residue was purified by column chromatography (2:3 ethyl acetate: hexanes) to yield hydrazine 3 as a colorless oil.

(S)-tert-butyl 2-(2-ethoxy-2-oxoethyl)-2-(1-methoxy-1-oxo-3-phenylpropan-2-yl)hydrazine carboxylate (3 a). R_{f}=0.6 (2:3 ethyl acetate:hexanes); 90% yield. ¹H NMR (400 MHz, CDCl₃) δ 1.23 (t, J=7.2 Hz, 3H), 1.44 (s, 9H), 3.01 (dd, J=13.6 and 9.4 Hz, 1H), 3.14 (dd, J=13.2 and 5.2 Hz, 1H), 3.55 (s, 3H), 3.64-3.85 (m, 3H), 4.12 (q, J=6.8 Hz, 2H), 6.95 (br s, 1H), 7.17-7.28 (m, 5H). ¹³C NMR (100 MHz, CDCl₃) δ 14.35, 28.50, 36.83, 51.77, 61.11, 69.02, 80.53, 126.85, 128.60, 129.46, 137.31, 172.06. ESI-MS 403.1 [M+Na]⁺.

### Example 3B

### Hydrazine Formation

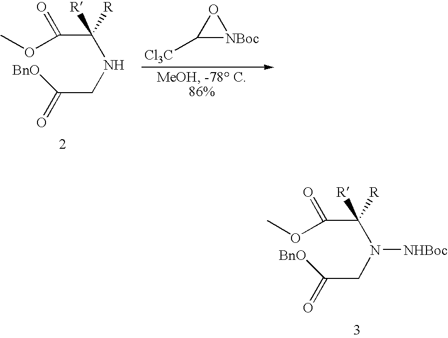

A solution of diester 2a (1.83 mmol) in dry methanol (18 mL) was cooled to −78° C. and kept under argon atmosphere. Tert-butyl 3-(trichloromethyl)-1,2-oxaziridine-2-carboxylate (2.38 mmol, 1.3 equiv.) was added slowly and the reaction was stirred at −78° C. for 4 hours, then at room temperature for 18 h. Progress of the reaction was monitored by TLC. The solvent was evaporated under reduced pressure and the crude residue was purified by column chromatography (2:3 ethyl acetate: hexanes) to yield hydrazine 3a as a thick colorless oil, which crystallized upon complete solvent evaporation.

### Example 4

### Diacid Formation

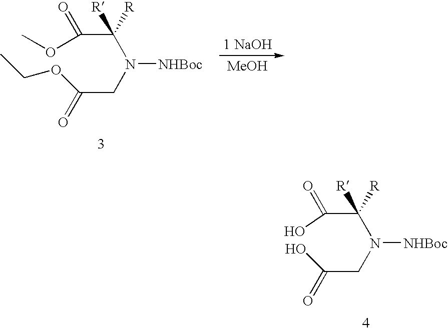

Hydrazine compound 3 (2.10 mmol) was dissolved in MeOH (12 mL) and cooled to 0° C. Then 1N NaOH (6.3 mmol, 3 equiv.) was added and stirred at room temperature overnight. MeOH was removed in vacuo and the residue was diluted with water. The aqueous solution was acidified with 1.2 N HCl, pH=1-2, and extracted with ethyl acetate (3×20 mL). The combined organic layers were washed with brine, dried over Na₂SO₄, and removed in vacuo to yield 4 as a white solid.

(S)-2-(2-(tert-butoxycarbonyl)-1-(carboxymethyl)hydrazinyl)-3-phenylpropanoic acid (4a). 75% yield. ¹H NMR (400 MHz, CD₃OD) δ 1.46 (s, 9H), 3.02 (d, J=7.2 Hz, 2H), 3.71 (s, 2H), 3.90 (t, J=7.2 Hz, 1H), 7.17-7.26 (m, 5H). ¹³C NMR (100 MHz, CD₃OD) δ 27.36, 35.96, 57.20, 69.26, 80.83, 126.41, 128.21, 129.04, 137.60, 157.20, 172.70, 173.41. ESI-MS 337.1 [M-H]⁺.

### Example 5

### Monoester Formation

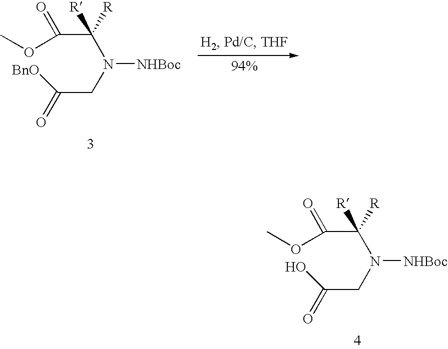

A solution of hydrazine 3a (0.45 mmol) in THF (5 mL) was placed in a hydrogenation vessel along with the Pd/C catalyst (10% mol). Hydrogenolysis of 3a took place at 35 psi, at room temperature for 30 min. Upon complete consumption of starting material 3a, indicated by TLC, the catalyst was filtered through celite and washed with THF. The filtrate was concentrated under reduced pressure to yield monoester 4a in quantitative yield.

### Example 6

### Synthesis of Protected Beta Alanine

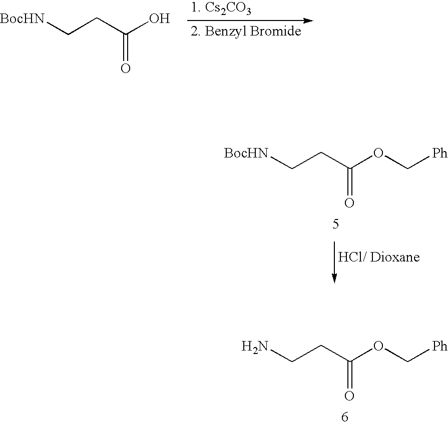

The benzyl protection was performed in one reaction. Boc-β-alanine was completely dissolved in anhydrous THF, and then cesium carbonate, 2 equiv., was added to this mixture and allowed to stir for 10 minutes. After that time, benzyl bromide, 3 equiv., was added to the mixture and stirred at room temperature overnight. This produced the pure compound 5, which was a clear oil in 97% yield.

Compound 5 was then treated with standard HCl/Dioxane conditions to remove the Boc group and form the salt. Now in the salt state, compound 6 could be used in the cyclization reactions.

### Example 7

### Hydrazine Amide Formation

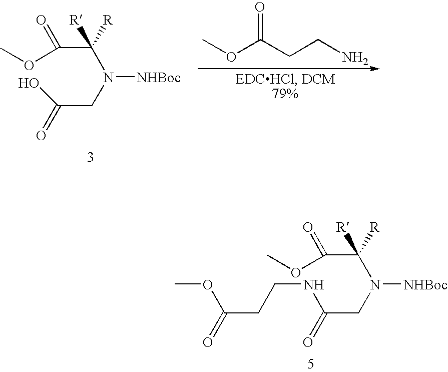

A mixture of monoester 4a (0.25 g, 0.70 mmol) and the HCl salt of β-alanine methyl ester (0.11 g, 0.78 mmol) in dry DCM (10 mL) was cooled to 0° C. under argon. EDC.HCl (0.16 g, 0.84 mmol) was added portionwise, while the mixture stirred vigorously. The reaction was kept at 0° C. for 30 min, then at room temperature for 16 hr. The mixture was quenched with saturated NaHCO₃ and extracted with CHCl₃ (3×5 mL). The combined organic layers were washed with 1N HCl, water, and brine; then dried over MgSO₄. The solvent was removed under vacuo and the obtained crude oil was purified by column chromatography (1:1 ethyl acetate: hexanes) to yield amide 5 in 79% yield.

### Example 8

### Hydrazine-Linked Mimetic Synthesis

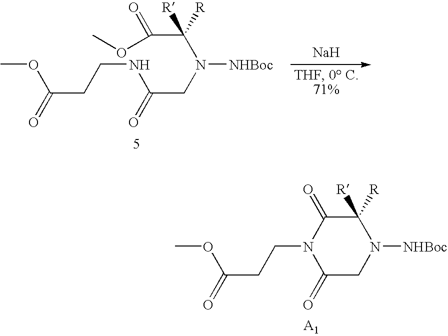

Compound 5 (0.34 mmol) was dissolved in dry THF (5 mL) and cooled to 0° C. under argon. To this mixture, a suspension of NaH (0.324 mmol) in THF (1 mL) was added dropwise. The reaction was kept at 0° C. for 30 min, then at room temperature for 22 hr. The solvent was removed under reduced pressure and the obtained crude was purified by column chromatography (8:2 ethylacetate: hexanes). Typical yields were 71%.

### Example 9

### Cyclization (Solution Phase Synthesis)

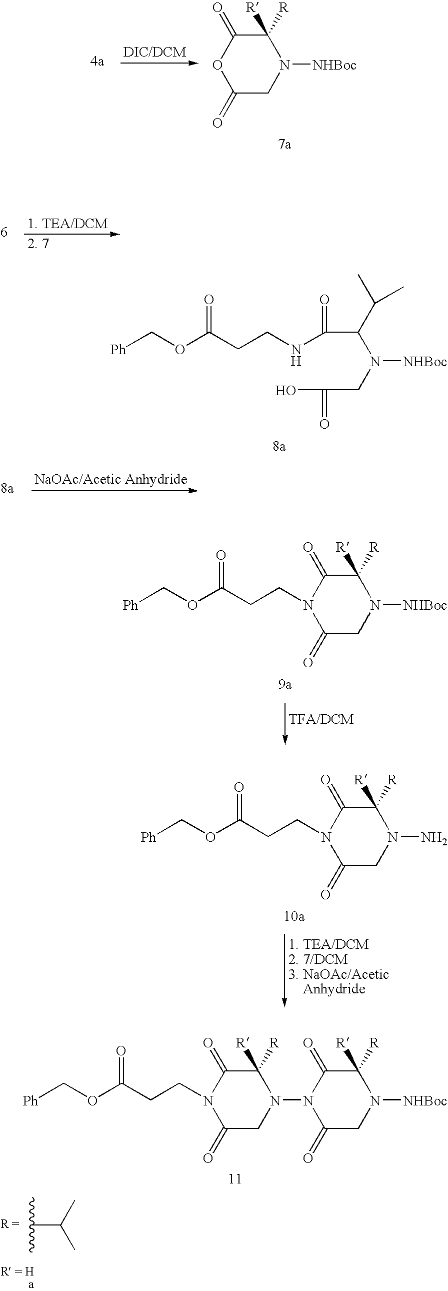

The cyclization process began with diacid 4a being dissolved in anhydrous dichloromethane (DCM). Diisopropycarbodimide (DIC), 1.1 equiv., was added drop wise to the mixture. The solution then stirred at room temperature for 3 hours. This produced compound 7a, which was not isolated due to its instability, but instead was kept in solution so it could be immediately used in the next step of the cyclization process.

For the following steps of this process, compound 6 was dissolved in anhydrous dichloromethane (DCM). Triethylamine, 3 eq., was added to the mixture and stirred for an additional 3 minutes. Quickly and carefully, compound 7a, 1 equiv., (which was still in solution and just finished the 3 hours of reacting time) was filtered into the mixture containing compound 6, DCM, and triethylamine This mixture was then refluxed at 47° C. for 4 hours, brought to room temperature, and stirred overnight. This produced compound 8a with a 33% yield.

Compound 8a was dissolved in acetic anhydride, sodium acetate, and 6 equiv. was added to the mixture, and then refluxed at 70° C. overnight to produce compound 9a in 55% yield. It is theorized that the low yield may be due to side reactions induced by the acetic anhydride.

Compound 9a was treated by standard TFA/DCM processes for deprotection. The salt product 10a could then be used to repeat the process over again, with each cycle adding to the scaffold 11. The β-alanine compound, 6, is only used for the first cycle of the scaffold formation process. Subsequent to that first cycle, variations of compound 7 (differing in R and R′ groups) were used in the synthesis.

A variety of compounds were successfully synthesized using Phe, Val, and Leu methyl ester hydrochlorides, for example, as the starting materials (see also Example 2B). A larger variety of amino compounds, both standard and non-standard, will be experimentally employed.

### Example 10

### Cyclization (Solid Phase Synthesis)

The cyclization described above in Example 9 was repeated in this Example using solid phase synthesis techniques. According to this procedure, the diacid (1 equiv.) was dissolved in minimum amount of NMP and added to a commercially available resin. DCC (4 equiv.), HOBt (4 equiv.) and DIEA (8 equiv) was dissolved in NMP and added to the resin.

The reaction mixture was bubbled with argon overnight. The resin was washed with NMP (3×), DCM (3×), MeOH (3×) and ether (3×).

For cyclization, the resin was swelled in DCE for 1 hr. Excess DCE was removed and acetic anhydride was added, followed by sodium acetate (6.6 equiv.). The reaction mixture was refluxed overnight at 90° C. The resin was washed with NMP (3×), DCM (3×), MeOH (3×) and ether (3×).

### Example 11

### Biological Testing

Bak BH3 peptide (GQVGRQLAIIGDDINR) was labeled with succinimidyl Oregon Green (Molecular Probes) and purified by HPLC. For the assays, 5 nM labeled BH3 peptide, 300 nM GST-Bcl-xl protein, mixed with PBS, pH 7.4 (Gibco), were added to 96-well EIA black plates (Costar #3694). Various concentrations of the mimetic formed in the above examples (e.g., containing Phe, Val, and Leu side chains) was transferred either manually or using plastic 96-pin arrays (Beckman). The plates were incubated for 15-30 min at 25° C., and FP values (mP) were determined with an Analyst plate reader (PerkinElmer).

Results are illustrated in Table 2:
BH3M-PD1

| | uM | Expt-I | uM | Expt-II | uM | Expt-III |
|---|---|---|---|---|---|---|
| | 0 | 184 | 0 | 172 | 0 | 169 |
| | 0.1 | 172 | 0.1 | 161 | 0.1 | 151 |
| | 1 | 155 | 1 | 158 | 1 | 154 |
| | 2 | 147 | 2 | 121 | 2 | 124 |
| | 5 | 99 | 5 | 76.2 | 5 | 84.1 |
| | 10 | 40.4 | 10 | 55.5 | 10 | 58.1 |
| | 20 | 22.8 | 20 | 7.2 | 20 | 12.3 |
| IC50 | | 5 uM | | 4 uM | | 5 uM |

IC50 = 4.67 ± 0.58

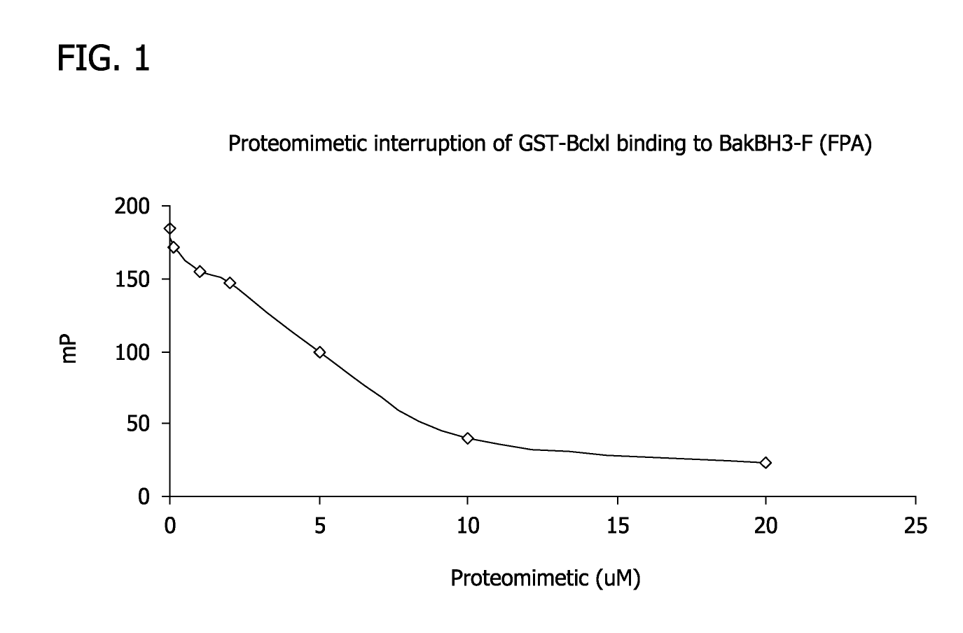

As shown in FIG. 1, the mimetic composition produced in the above examples interrupts GST-Bcl-x_{L} binding to Bak BH3-F (FPA).

What is claimed is:

1. A composition having the formula Z₁—(W₁)ₙ—P₁-P₂—(W₂)^{m}—Z₂ wherein P₁ and P₂ are six-membered heterocyclic 1,4-diaza-2-one moieties covalently bonded by a hydrazine bond;
each W₁ and W₂ is independently a six-membered carbocyclic or heterocyclic ring;
Z₁ and Z₂ are independently hydrogen, hydrocarbyl, substituted hydrocarbyl, heterocyclo, acyl, aryl, amino, protected amino, sulfonyl, phosphoryl, or a linker moiety attached to a solid support; and
n is 0 to 10 and m is 0 to 10 provided, however, the sum of n and m is 1 to 10.

2. The composition of claim 1 wherein the six-membered heterocyclic 1,4-diaza-2-one moieties covalently bonded by a hydrazine bond correspond to Formula (50): 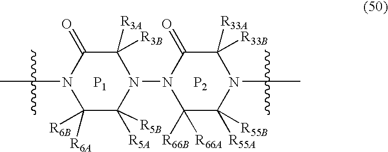
wherein
R_{3A}, R_{3B}, R_{33A}, and R_{33B} are independently hydrogen, substituted or unsubstituted hydrocarbyl, heterocyclo, alkoxy, ester, thioester, thioether, amido, or amino;
R_{5A}, R_{5B}, R_{55A}, and R_{55B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O;
R_{6B} is hydrogen or together with R_{6A} forms ═O;
R_{66A} is hydrogen, sulfonyl, or phosphoryl, or together with R_{66B} forms ═O; and
R_{66B} is hydrogen or together with R_{66A} forms ═O.

3. The composition of claim 2 wherein R_{3A}, R_{3B}, R_{33A}, and R_{33B} are independently hydrogen, hydrocarbyl, or substituted hydrocarbyl.

4. The composition of claim 2 wherein R_{3A}, R_{3B}, R_{33A}, and R_{33B} are independently hydrogen, alkanoic acid, alkanol, alkyl, substituted alkyl, alkylene amide, alkylene amine, aralkyl, substituted aralkyl, or heteroaralkyl.

5. The composition of claim 2 wherein at least one of R_{3A}, R_{3B}, R_{33A}, and R_{33B} is other than hydrogen.

6. The composition of claim 3 wherein R_{5A}, R_{5B}, R_{55A}, and R_{55B} are hydrogen.

7. The composition of claim 3 wherein R_{6A} and R_{6B} together form ═0, and R_{66A} and R_{66B} together form ═O.

8. The composition of claim 2 wherein the sum of m and n is 1 or 2.

9. The composition of claim 1 wherein the six-membered heterocyclic 1,4-diaza-2-one moieties covalently bonded by a hydrazine bond correspond to Formula (500): 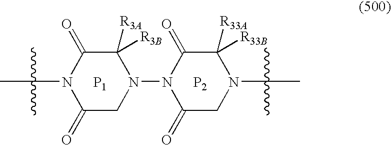
wherein
R_{3A}, R_{3B}, R_{33A}, and R_{33B} are independently hydrogen, hydrocarbyl, or substituted hydrocarbyl.

10. The composition of claim 9 wherein R_{3A}, R_{3B}, R_{33A}, and R_{33B} are independently hydrogen, alkanoic acid, alkanol, alkyl, substituted alkyl, alkylene amide, alkylene amine, aralkyl, substituted aralkyl, or heteroaralkyl.

11. The composition of claim 9 wherein at least one of R_{3A}, R_{3B}, R_{33A}, and R_{33B} is other than hydrogen.

12. The composition of claim 2 wherein each W₁ and W₂ corresponds to Formula (60): 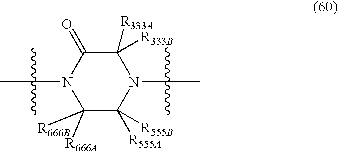
wherein
R_{333A} and R_{333B} are independently hydrogen, substituted or unsubstituted hydrocarbyl, heterocyclo, alkoxy, ester, thioester, thioether, amido, or amino;
R_{555A} and R_{555B} are independently hydrogen, hydrocarbyl, substituted hydrocarbyl, or together with the carbon atom to which they are attached form a five- or six-membered ring;
R_{666A} is hydrogen, sulfonyl, or phosphoryl, or together with R_{666B} forms ═O; and
R_{666B} is hydrogen or together with R_{666A} forms ═O.

13. The composition of claim 12 wherein R_{333A} and R_{333B} are independently hydrogen, hydrocarbyl, or substituted hydrocarbyl.

14. The composition of claim 12 wherein R_{333A} and R_{333B} are independently hydrogen, alkanoic acid, alkanol, alkyl, substituted alkyl, alkylene amide, alkylene amine, aralkyl, substituted aralkyl, or heteroaralkyl.

15. The composition of claim 12 wherein at least one of R_{333A} and R_{333B} is other than hydrogen.

16. The composition of claim 13 wherein R_{555A} and R_{555B} are hydrogen.

17. The composition of claim 13 wherein R_{666A} and R_{666B} together form ═O.

18. The composition of claim 9 wherein each W₁ and W₂ corresponds to Formula (600): 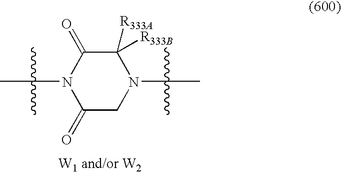
wherein
R_{333A} and R_{333B} are independently hydrogen, substituted or unsubstituted hydrocarbyl, heterocyclo, alkoxy, ester, thioester, thioether, amido, or amino

19. The composition of claim 18 wherein R_{333A} and R_{333B} are independently hydrogen, hydrocarbyl, or substituted hydrocarbyl.

20. The composition of claim 18 wherein R_{333A} and R_{333B} are independently hydrogen, alkanoic acid, alkanol, alkyl, substituted alkyl, alkylene amide, alkylene amine, aralkyl, substituted aralkyl, or heteroaralkyl.

21. The composition of claim 18 wherein at least one of R_{333A} and R_{333B} is other than hydrogen.

22. The composition of claim 2 wherein
Z₁ is a linker moiety attached to a solid support; and
Z₂ is hydrogen, hydrocarbyl, substituted hydrocarbyl, heterocyclo, acyl, amino, protected amino, sulfonyl, or phosphoryl.

23. The composition of claim 2 wherein Z₁ and Z₂ are independently hydrogen, hydrocarbyl, substituted hydrocarbyl, heterocyclo, acyl, amino, protected amino, sulfonyl, or phosphoryl.

24. A process for the preparation of a composition containing two, six-membered heterocyclic 1,4-diaza-2-one moieties covalently bonded by a hydrazine bond, the process comprising treating a six-membered heterocyclic 1,4-diaza-4′-amino-2-one with a monoester (44a) or a diacid (44b) in the presence of an activating agent, wherein:
the monoester (44a) and the diacid (44b) correspond to Formulae (44a) or (44b): 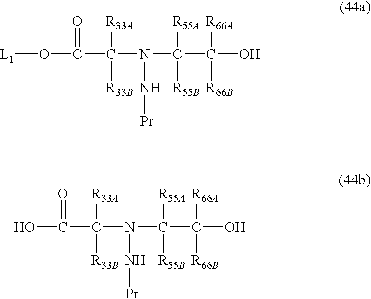
L₁ is hydrocarbyl or substituted hydrocarbyl;
Pr is an amino protecting group;
R_{3A}, R_{3B}, R_{33A}, and R_{33B} are independently hydrogen, substituted or unsubstituted hydrocarbyl, heterocyclo, alkoxy, ester, thioester, thioether, amino, or amido;
R_{55A} and R_{55B} are independently hydrogen, hydrocarbyl, substituted hydrocarbyl, or together with the carbon atom to which they are attached form a five- or six-membered ring;
R_{66A} is hydrogen, sulfonyl, or phosphoryl, or together with R_{66B} forms ═O; and
R_{66B} is hydrogen or together with R_{66A} forms ═O.

25. The process of claim 24 wherein the two, six-membered heterocyclic 1,4-diaza-2-one moieties covalently bonded by a hydrazine bond correspond to Formula (51): 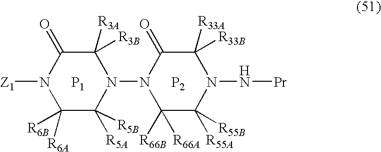
wherein
Pr is an amino protecting group;
R_{3A}, R_{3B}, R_{33A}, and R_{33B} are independently hydrogen, substituted or unsubstituted hydrocarbyl, heterocyclo, alkoxy, ester, thioester, thioether, amino, or amido;
R_{5A}, R_{5B}, R_{55A}, and R_{55B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O;
R_{6B} is hydrogen or together with R_{6A} forms ═O;
R_{66A} is hydrogen, sulfonyl, or phosphoryl, or together with R_{66B} forms ═O;
R_{66B} is hydrogen or together with R_{66A} forms ═O; and
Z₁ is hydrogen, hydrocarbyl, substituted hydrocarbyl, heterocyclo, acyl, aryl, amino, protected amino, sulfonyl, phosphoryl, or a linker moiety attached to a solid support.

26. The process of claim 25 further comprising treating the two, six-membered heterocyclic 1,4-diaza-2-one moieties covalently bound by a hydrazine bond with a monoester (444a) or a diacid (444b) in the presence of an activating agent to form a composition containing three, six-membered heterocyclic 1,4-diaza-2-one moieties covalently bonded by hydrazine bonds, wherein
the monoester (444a) or diacid (444b) correspond to Formulae (444a) and (444b), respectively: 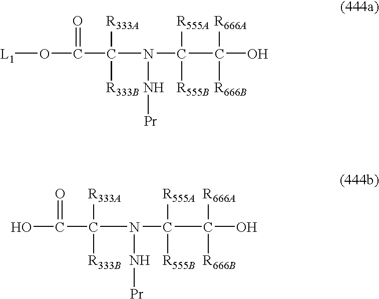
L₁ is hydrocarbyl or substituted hydrocarbyl;
Pr is an amino protecting group;
R_{333A} and R_{333B} are independently hydrogen, substituted or unsubstituted hydrocarbyl, heterocyclo, alkoxy, ester, thioester, thioether, amino, or amido;
R_{555A} and R_{555B} are independently hydrogen, hydrocarbyl, substituted hydrocarbyl, or together with the carbon atom to which they are attached form a five- or six-membered ring;
R_{666A} is hydrogen, sulfonyl, or phosphoryl, or together with R_{666B} forms ═O; and
R_{666B} is hydrogen or together with R_{666A} forms ═O.

27. The process of claim 26 wherein the three, six-membered heterocyclic 1,4-diaza-2-one moieties covalently bonded by hydrazine bonds corresponds to Formula (52): 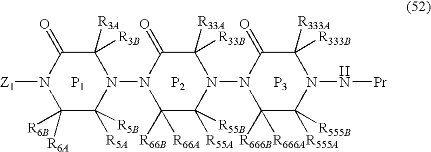
wherein
Pr is an amino protecting group;
R_{3A}, R_{3B}, R_{33A}, R_{33B}, R_{333A}, and R_{333B} are independently hydrogen, substituted or unsubstituted hydrocarbyl, heterocyclo, alkoxy, ester, thioester, thioether, amino, or amido;
R_{5A}, R_{5B}, R_{55A}, R_{55B}, R_{555A}, and R_{555B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O;
R_{6B} is hydrogen or together with R_{6A} forms ═O;
R_{66A} is hydrogen, sulfonyl, or phosphoryl, or together with R_{66B} forms ═O;
R_{66B} is hydrogen or together with R_{66A} forms ═O;
R_{666A} is hydrogen, sulfonyl, or phosphoryl, or together with R_{666B} forms ═O;
R_{666B} is hydrogen or together with R_{666A} forms ═O; and
Z₁ is hydrogen, hydrocarbyl, substituted hydrocarbyl, heterocyclo, acyl, aryl, amino, protected amino, sulfonyl, phosphoryl, or a linker moiety attached to a solid support.

28. The process of claim 24 wherein the six-membered heterocyclic 1,4-diaza-4′-amino-2-one corresponds to Formula (60): 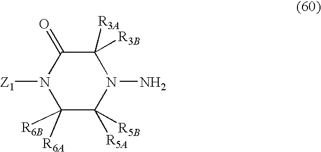
R_{3A} and R_{3B} are independently hydrogen, substituted or unsubstituted hydrocarbyl, heterocyclo, alkoxy, ester, thioester, thioether, amino, or amido;
R_{5A} and R_{5B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O;
R_{6B} is hydrogen or together with R_{6A} forms ═O; and
Z₁ is hydrogen, hydrocarbyl, substituted hydrocarbyl, heterocyclo, acyl, aryl, amino, protected amino, sulfonyl, phosphoryl, or a linker moiety attached to a solid support.

29. The process of claim 28 wherein the six-membered heterocyclic 1,4-diaza-4′-amino-2-one corresponding to Formula (10A) is prepared by treating a monoester (4a) or a diacid (4b) with an activating agent, wherein the monoester (4a) or diacid (4b) correspond to Formulae (4a) and (4b), respectively: 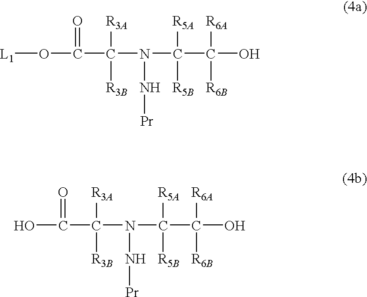
wherein
Pr is an amino protecting group;
R_{3A} and R_{3B} are independently hydrogen, substituted or unsubstituted hydrocarbyl, heterocyclo, alkoxy, ester, thioester, thioether, amino, or amido;
R_{5A} and R_{5B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O; and
R_{6B} is hydrogen or together with R_{6A} forms ═O.

30. The process of claim 29 wherein the monoester (4a) or diacid (4b) is prepared by treating an protected hydrazine (3) with a hydrolyzing agent, wherein the protected hydrazine (3) corresponds to Formula (3): 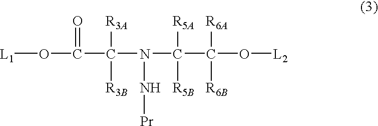
L₁ and L₂ are independently hydrogen, hydrocarbyl, or substituted hydrocarbyl provided, however, at least one of L₁ and L₂ is not hydrogen;
Pr is an amino protecting group;
R_{3A} and R_{3B} are independently hydrogen, substituted or unsubstituted hydrocarbyl, heterocyclo, alkoxy, ester, thioester, thioether, amino, or amido;
R_{5A} and R_{5B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O; and
R_{6B} is hydrogen or together with R_{6A} forms ═O.

31. The process of claim 30 wherein the protected hydrazine (3) is prepared by introducing a protected amino moiety (—NH—Pr) to a secondary amine (2), wherein the secondary amine (2) corresponds to Formula (2): 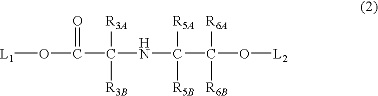
L₁ and L₂ are independently hydrogen, hydrocarbyl, or substituted hydrocarbyl provided, however, at least one of L₁ and L₂ is not hydrogen;
R_{3A} and R_{3B} are independently hydrogen, substituted or unsubstituted hydrocarbyl, heterocyclo, alkoxy, ester, thioester, thioether, amino, or amido;
R_{5A} and R_{5B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O; and
R_{6B} is hydrogen or together with R_{6A} forms ═O.

32. The process of claim 31 wherein the protected amino moiety (—NH—Pr) is introduced to the secondary amine (2) by a process comprising the steps of:
(a) nitrosating the secondary amine (2), reducing the resulting nitro moiety to form an amino moiety, and protecting the amino moiety with an amino protecting group; or
(b) treating the secondary amine (2) with an N-protected oxaziridine.

33. The process of claim 32 wherein the secondary amine (2) is prepared by treating an amino acid or ester thereof (1) with an alkylating agent (1A), wherein
the amino acid or ester thereof (1) corresponds to Formula (1): 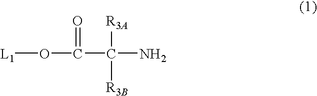
the alkylating agent (1A) corresponds to Formula (1A): 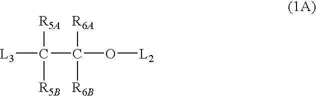
L₁ and L₂ are independently hydrogen, hydrocarbyl, or substituted hydrocarbyl provided, however, at least one of L₁ and L₂ is not hydrogen;
L₃ is halo;
R_{3A} and R_{3B} are independently hydrogen, substituted or unsubstituted hydrocarbyl, heterocyclo, alkoxy, ester, thioester, thioether, amino, or amido;
R_{5A} and R_{5B} are independently hydrogen, hydrocarbyl, substituted hydrocarbyl, or together with the carbon atom to which they are attached form a five- or six-membered ring;
R_{6A} is hydrogen, sulfonyl, or phosphoryl, or together with R_{6B} forms ═O; and
R_{6B} is hydrogen or together with R_{6A} forms ═O.

34. The process of claim 27 further comprising (i) removing the amino protecting group (—Pr) from the three, six-membered heterocyclic 1,4-diaza-2-one moieties covalently bonded by hydrazine bonds and (ii) derivatizing the resulting amino moiety, the derivatization comprising treating the deprotected three, six-membered heterocyclic 1,4-diaza-2-one moieties covalently bonded by hydrazine bonds with L₄-Z₂, wherein
L₄ is a leaving group; and
Z₂ is hydrogen, hydrocarbyl, substituted hydrocarbyl, heterocyclo, acyl, aryl, amino, protected amino, sulfonyl, or phosphoryl,

35. The process of claim 27 wherein Z₁ is a linker moiety attached to a solid support, the process further comprising (i) treating the three, six-membered heterocyclic 1,4-diaza-2-one moieties covalently bonded by hydrazine bonds with a hydrolyzing agent to remove the linker moiety attached to a solid support and (ii) derivatizing the resulting —NH moiety, the derivatization comprising treating the three, six-membered heterocyclic 1,4-diaza-2-one moieties covalently bonded by hydrazine bonds with L₄-Z₁, wherein,
L₄ is a leaving group; and
Z₁ is hydrogen, hydrocarbyl, substituted hydrocarbyl, heterocyclo, acyl, aryl, amino, protected amino, sulfonyl, or phosphoryl.
